(19) United States
(12) Patent Application Publication
(10) Pub. No.: US 2026/0345097 A1
(43) Pub. Date: Nov. 5, 2026

(54) FLOOR PLAN MAPPING SYSTEM FOR REAL-TIME DEVICE LOCATION TRACKING

(71) Applicant: HLFIP Holding, LLC, Atlanta, GA (US)

(72) Inventor: Jonathan D. Logan, St. Petersburg, FL (US)

(73) Assignee: HLFIP Holding, LLC, Atlanta, GA (US)

(21) Appl. No.: 19/744,512

(22) Filed: Jul. 16, 2026

Related U.S. Application Data
(63) Continuation-in-part of application No. 19/217,435, filed on May 23, 2025.
Application No. 19/217,435 is a continuation of application No. 17/580,462, filed on Jan. 20, 2022, now Pat. No. 12,310,762.
(60) Provisional application No. 63/139,778, filed on Jan. 20, 2021.

(51) Int. Cl. A61B 5/00

(52) U.S. Cl. CPC A61B 5/746; A61B 5/0022; A61B 5/681; A61B 5/7405; A61B 5/742; A61B 5/7465

(57) ABSTRACT

A computer-implemented system and method for real-time indoor personnel tracking within a facility. The system receives a digital floor plan image and enables definition of geographic zones through polygon overlays designating rooms, corridors, and restricted areas. Virtual beacon placement markers are positioned on the floor plan corresponding to physical ultra-wideband wireless beacon locations throughout the facility. The system communicates with wearable devices carried by personnel to determine location coordinates, including vertical positioning for multi-level structures. A graphical interface displays real-time personnel positions superimposed on the floor plan. The system provides automated occupancy counting, movement history logging, and alert generation when individuals enter designated keep-away zones. The invention enables facility managers to monitor personnel distribution, enforce access restrictions, and maintain accurate headcounts through integration of floor plan mapping, beacon infrastructure configuration, and precision indoor positioning technology.

### CROSS-REFERENCE TO RELATED APPLICATIONS

This application is a continuation-in-part of U.S. Patent Application 19/217,435, filed May 23, 2025, titled “SMART WEARABLE DEVICE FOR TRACKING AND MONITORING INDIVIDUALS IN A CORRECTIONAL FACILITY” which is a continuation of U.S. Patent Application 17/580,462, filed Jan. 20, 2022, titled “SMART WEARABLE DEVICE FOR TRACKING AND MONITORING INDIVIDUALS IN A CORRECTIONAL FACILITY.” which claims the benefit of U.S. Provisional Application 63/139,778, filed Jan. 20, 2021, titled “SMART WEARABLE DEVICE FOR TRACKING AND MONITORING INDIVIDUALS IN A CORRECTIONAL FACILITY.” The contents of the above identified applications are incorporated herein by reference in their entirety.

### BACKGROUND

### FIELD OF THE ART

The present invention relates generally to indoor location tracking systems, and more particularly to a computer-implemented system and method for real-time tracking of individuals within a facility using beacon-based positioning technology integrated with smart wearable devices and interactive floor plan mapping.

### Discussion of the State of the Art

Facilities including correctional institutions, hospitals, and large commercial buildings require the ability to track the real-time locations of individuals within their premises. Such tracking serves multiple operational needs, including locating specific individuals during emergencies, verifying compliance with movement restrictions, and maintaining accurate population counts across different areas of a facility.

Traditional approaches to indoor personnel tracking have relied on manual check-ins, periodic headcounts, and basic access control systems. Manual check-ins require individuals to report their presence at designated stations, creating gaps in location awareness between check-in events. Periodic headcounts provide only intermittent snapshots of population distribution and require staff resources to execute. Basic access control systems record passage through doors and controlled entry points but provide no information about an individual’s location between such transition events. These methods share a common limitation in that they cannot provide continuous location awareness throughout a facility.

Previous solutions have attempted to address indoor tracking through GPS-based systems. However, GPS technology is fundamentally limited in indoor environments because satellite signals cannot penetrate building structures with sufficient reliability or accuracy. The signal degradation that occurs when GPS attempts to operate through roofs, walls, and floors renders such systems impractical for determining indoor positions with the precision required for room-level location identification.

WiFi signal strength triangulation represents another approach that has been applied to indoor positioning. These systems analyze the received signal strength from multiple WiFi access points to estimate a device’s position. However, WiFi-based positioning systems typically achieve accuracy only within a range of fifteen to thirty feet. This level of precision is insufficient for determining which specific room within a facility a person occupies, particularly in environments with smaller rooms or areas where precise location determination is necessary for operational purposes.

RFID badge reader systems have also been deployed for indoor tracking applications. These systems detect the presence of RFID-enabled badges when individuals pass within range of fixed reader units. However, RFID systems only provide location data at the discrete chokepoints where readers are installed. Areas between reader locations remain unmonitored, creating blind spots throughout facilities. This architecture fails to provide the continuous tracking necessary to know an individual’s location at arbitrary points in time.

Beyond the limitations of the underlying positioning technologies, existing systems present challenges in their configuration and deployment. Setting up indoor tracking systems requires extensive manual configuration to map physical spaces to digital coordinate systems. Technical specialists must translate architectural dimensions and room layouts into coordinate frameworks that the tracking system can interpret. No standardized method exists for converting architectural floor plans into functional tracking zones that can be associated with beacon hardware and wearable devices.

Existing systems also lack intuitive tools for facility administrators to define tracking zones, assign location categories, or designate beacon placements. The configuration process typically requires specialized technical knowledge, limiting the ability of facility operations staff to modify or expand tracking coverage without external support.

Additionally, current systems fail to provide integration between floor plan visualization and real-time position data. Operators using these systems must mentally translate between abstract coordinate data presented by the tracking system and the physical locations those coordinates represent within the facility. This disconnect between data presentation and physical space comprehension reduces the utility of the tracking information during time-sensitive situations when rapid location identification is required.

### SUMMARY

Conventional indoor location tracking systems have presented several limitations that reduce their effectiveness in facility management applications. Prior solutions typically rely on GPS technology, which provides inadequate accuracy for indoor environments due to signal attenuation from building structures. Existing WiFi-based positioning systems offer location accuracy measured in tens of feet, which proves insufficient for applications requiring precise individual tracking within defined zones and rooms. Additionally, prior systems often require substantial manual configuration and lack integration between floor plan visualization and location determination components, resulting in fragmented workflows for facility managers.

Previous approaches to facility monitoring have also failed to provide adequate tools for defining and managing restricted areas, requiring separate systems for zone management and location tracking. The lack of three-dimensional positioning in conventional systems further limits their utility in facilities with multi-level spaces such as mezzanines, balconies, and split-level areas where vertical position determination is required.

The present invention provides a computer-implemented system for indoor location tracking that transforms static floor plan images into dynamic, interactive tracking interfaces with beacon-based position determination. The system comprises several integrated components that operate together to provide real-time location awareness within a facility.

In a first aspect, the invention provides a method for establishing a tracking-enabled floor plan. The method includes importing floor plan data from standard image file formats into a computer system and establishing a global coordinate origin at a predetermined location, such as the top-left corner of the floor plan image. This coordinate origin serves as the reference point for all subsequent spatial calculations performed by the system. The method further includes calibrating the floor plan to real-world scale by receiving user input specifying known dimensions of the depicted space while maintaining aspect ratio integrity of the original image.

In a second aspect, the invention provides a polygon-based zone definition interface. Users draw polygons directly on the floor plan through a graphical user interface, with each polygon defining a distinct location area corresponding to physical spaces such as rooms, hallways, zones, or exterior locations. The system includes a drag-and-drop interface for assigning location categories to defined polygons, enabling classification of areas according to their function, including housing units, restricted zones, and keep-away areas where designated individuals are prohibited from entering.

In a third aspect, the invention provides a beacon placement optimization component. The system generates recommended beacon placement locations based on the defined floor plan geometry to provide coverage sufficient for locating any wearable device within the facility boundaries. Virtual markers indicating beacon positions are displayed on the floor plan interface, and the system includes a calibration feature for adjusting beacon positions after physical installation to account for differences between planned and actual placements.

In a fourth aspect, the invention provides a real-time location determination engine utilizing ultra-wide band communication technology. Ultra-wide band beacons installed throughout the facility communicate with smart wearable devices at periodic intervals. The system processes signals received from multiple beacons to perform triangulation-based position calculations, achieving location accuracy within several feet or even a few inches. Device coordinates are calculated relative to the global coordinate origin established during floor plan configuration.

In a fifth aspect, the invention provides three-dimensional position determination by incorporating elevation data. The system determines not only horizontal position but also the height of wearable devices from the floor surface, enabling tracking in facilities with mezzanines, balconies, and other multi-level spaces within a single floor plan.

In a sixth aspect, the invention provides a visualization and monitoring interface. The system displays visual indicators overlaid on the floor plan showing real-time positions of tracked individuals. Multiple viewing modes are supported, including an Inmate View that automatically scrolls the floor plan display to maintain a selected individual in the visible area, and an Area View for monitoring all individuals within a selected zone. The system stores historical location data with associated zone, room, and beacon information, enabling movement tracking analysis and automated headcount generation.

In a seventh aspect, the invention provides a compliance monitoring and alert system. The system monitors keep-away restrictions by performing proximity calculations between multiple tracked devices. When a tracked individual enters a designated keep-away zone or comes within a specified proximity of another individual subject to a keep-away restriction, the system generates an alert notification.

In an eighth aspect, the invention provides integration capabilities with existing facility management infrastructure through a dashboard interface. The system includes configurable enable flags at both location and facility levels, allowing administrators to selectively activate tracking functionality for specific areas or entire facilities. A real-time ping functionality enables on-demand location queries in addition to the periodic tracking updates.

The present invention provides several improvements over prior indoor location tracking solutions. The integration of floor plan import, zone definition, beacon placement, and real-time tracking into a unified system eliminates the fragmented workflows associated with prior approaches that required multiple disparate systems.

The use of ultra-wide band technology provides location accuracy within approximately several feet or even a few inches, representing a substantial improvement over WiFi-based positioning systems that typically achieve accuracy measured in tens of feet. This increased accuracy enables reliable determination of which specific room or zone an individual occupies.

The polygon-based zone definition interface allows facility managers to define irregularly-shaped areas that correspond to actual room boundaries, rather than being limited to rectangular zones as in some prior systems. The drag-and-drop category assignment streamlines the configuration process.

The three-dimensional positioning capability addresses limitations of prior two-dimensional tracking systems by enabling accurate tracking in facilities with vertical complexity, including mezzanines and multi-level spaces.

The automated keep-away monitoring and alert generation provides compliance functionality that was previously achievable only through manual observation or separate monitoring systems.

The beacon calibration feature accommodates real-world installation variations, improving system accuracy without requiring precise adherence to planned beacon placements during physical installation.

The image optimization component reduces storage requirements while maintaining visual fidelity of floor plan displays, improving system efficiency for facilities with multiple floor plans or high-resolution source images.

### BRIEF DESCRIPTION OF THE DRAWINGS

The accompanying drawings illustrate several embodiments and, together with the description, serve to explain the principles of the invention according to the embodiments. It will be appreciated by one skilled in the art that the particular arrangements illustrated in the drawings are merely exemplary and are not to be considered as limiting of the scope of the invention or the claims herein in any way.

FIG. 1 illustrates a network architecture for Floor Plan Mapping System for Real-Time Device Location Tracking in accordance with an exemplary embodiment of the invention.

FIG. 2 illustrates a system architecture for Floor Plan Mapping System for Real-Time Device Location Tracking in accordance with an exemplary embodiment of the present invention.

FIG. 3 illustrates an exemplary computer implemented process for Floor Plan Mapping System for Real-Time Device Location Tracking according to one embodiment of the invention.

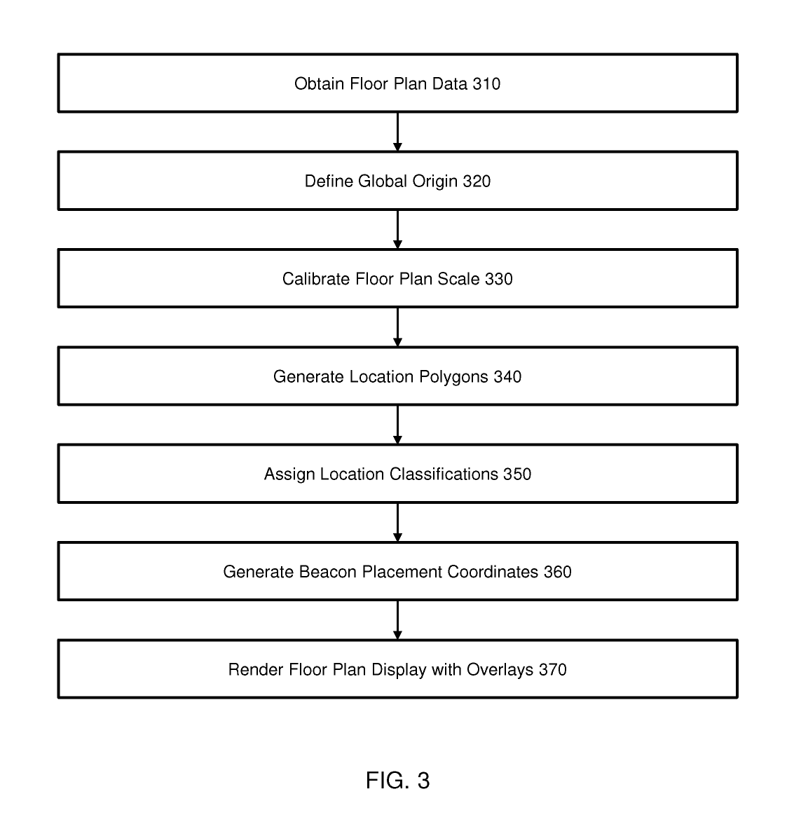

FIG. 4 illustrates one embodiment of the computing architecture that supports an embodiment of the inventive disclosure.

FIG. 5 illustrates components of a system architecture that supports an embodiment of the inventive disclosure.

FIG. 6 illustrates components of a computing device that supports an embodiment of the inventive disclosure.

FIG. 7 illustrates components of a computing device that supports an embodiment of the inventive disclosure.

### DETAILED DESCRIPTION

One or more different embodiments may be described in the present application. Further, for one or more of the embodiments described herein, numerous alternative arrangements may be described; it should be appreciated that these are presented for illustrative purposes only and are not limiting of the embodiments contained herein or the claims presented herein in any way. One or more of the arrangements may be widely applicable to numerous embodiments, as may be readily apparent from the disclosure. In general, arrangements are described in sufficient detail to enable those skilled in the art to practice one or more of the embodiments, and it should be appreciated that other arrangements may be utilized and that structural, logical, software, electrical and other changes may be made without departing from the scope of the embodiments. Particular features of one or more of the embodiments described herein may be described with reference to one or more particular embodiments or figures that form a part of the present disclosure, and in which are shown, by way of illustration, specific arrangements of one or more of the aspects. It should be appreciated, however, that such features are not limited to usage in the one or more particular embodiments or figures with reference to which they are described. The present disclosure is neither a literal description of all arrangements of one or more of the embodiments nor a listing of features of one or more of the embodiments that must be present in all arrangements.

Headings of sections provided in this patent application and the title of this patent application are for convenience only and are not to be taken as limiting the disclosure in any way.

Devices that are in communication with each other need not be in continuous communication with each other, unless expressly specified otherwise. In addition, devices that are in communication with each other may communicate directly or indirectly through one or more communication means or intermediaries, logical or physical.

A description of an aspect with several components in communication with each other does not imply that all such components are required. To the contrary, a variety of optional components may be described to illustrate a wide variety of possible embodiments and in order to more fully illustrate one or more embodiments. Similarly, although process steps, method steps, algorithms or the like may be described in a sequential order, such processes, methods and algorithms may generally be configured to work in alternate orders, unless specifically stated to the contrary. In other words, any sequence or order of steps that may be described in this patent application does not, in and of itself, indicate a requirement that the steps be performed in that order. The steps of described processes may be performed in any order practical. Further, some steps may be performed simultaneously despite being described or implied as occurring non-simultaneously (e.g., because one step is described after the other step). Moreover, the illustration of a process by its depiction in a drawing does not imply that the illustrated process is exclusive of other variations and modifications thereto, does not imply that the illustrated process or any of its steps are necessary to one or more of the embodiments, and does not imply that the illustrated process is preferred. Also, steps are generally described once per aspect, but this does not mean they must occur once, or that they may only occur once each time a process, method, or algorithm is carried out or executed. Some steps may be omitted in some embodiments or some occurrences, or some steps may be executed more than once in a given aspect or occurrence.

When a single device or article is described herein, it will be readily apparent that more than one device or article may be used in place of a single device or article. Similarly, where more than one device or article is described herein, it will be readily apparent that a single device or article may be used in place of the more than one device or article.

The functionality or the features of a device may be alternatively embodied by one or more other devices that are not explicitly described as having such functionality or features. Thus, other embodiments need not include the device itself.

Techniques and mechanisms described or referenced herein will sometimes be described in singular form for clarity. However, it should be appreciated that particular embodiments may include multiple iterations of a technique or multiple instantiations of a mechanism unless noted otherwise. Process descriptions or blocks in figures should be understood as representing modules, segments, or portions of code which include one or more executable instructions for implementing specific logical functions or steps in the process. Alternate implementations are included within the scope of various embodiments in which, for example, functions may be executed out of order from that shown or discussed, including substantially concurrently or in reverse order, depending on the functionality involved, as would be understood by those having ordinary skill in the art.

The detailed description set forth herein in connection with the appended drawings is intended as a description of various configurations and is not intended to represent the only configurations in which the concepts described herein may be practiced. The detailed description includes specific details for the purpose of providing a thorough understanding of various concepts. However, it will be apparent to those skilled in the art that these concepts may be practiced without these specific details. In some instances, well known structures and components are shown in block diagram form in order to avoid obscuring such concepts.

### Network Architecture

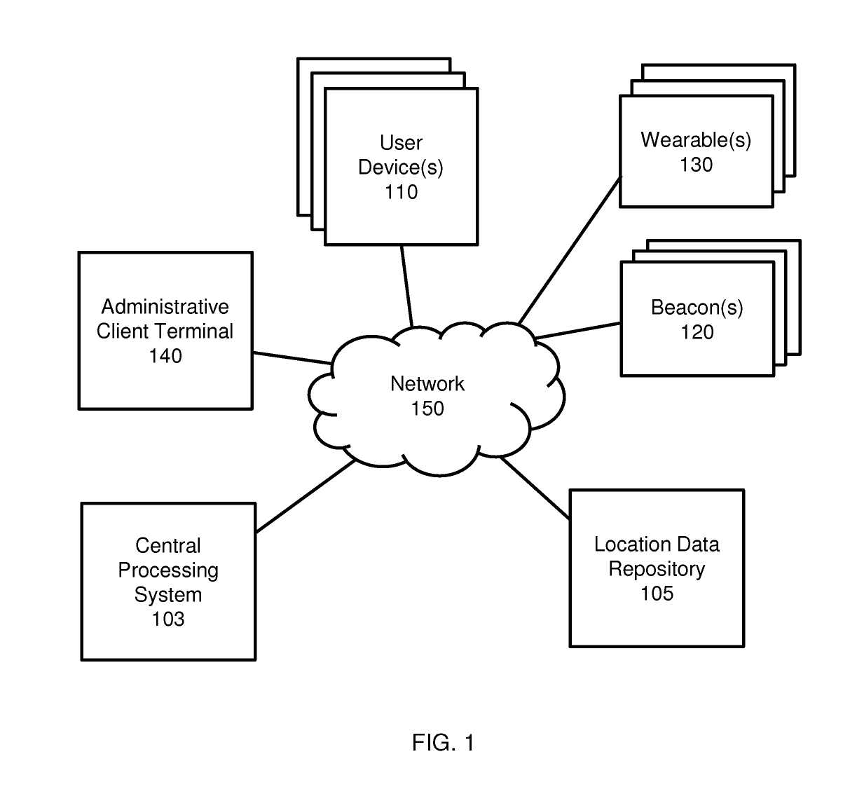

FIG. 1 illustrates an exemplary embodiment of Floor Plan Mapping System for Real-Time Device Location Tracking according to one embodiment. The network diagram includes: Central Processing System 103, Beacon(s) 120, Wearable User Device 130, Administrative Client Terminal 140, Location Data Repository 105, User Device(s) 110, and Network 150. The various components described herein are exemplary and for illustration purposes only and any combination or subcombination of the various components may be used as would be apparent to one of ordinary skill in the art. The system may be reorganized or consolidated, as understood by a person of ordinary skill in the art, to perform the same tasks on one or more other servers or computing devices without departing from the scope of the invention.

At a high level the Central Processing System 103 constitutes a computational infrastructure component that provides processing capabilities, data storage, and algorithmic execution for the indoor positioning and location classification system. The Central Processing System 103 operates as a backend computational resource that receives, processes, and analyzes data from multiple system components to enable accurate position determination and spatial classification within indoor environments.

The Central Processing System 103 performs several operational functions within the system architecture. The Central Processing System 103 processes floor plan data that has been imported into the system, converting graphical representations of indoor spaces into computationally usable coordinate information. The Central Processing System 103 calculates polygon coordinates that define spatial boundaries within the indoor environment, enabling the system to establish discrete zones and regions for location classification purposes. The Central Processing System 103 executes beacon signal triangulation algorithms that process signal data received from ultra-wide band beacons distributed throughout the monitored environment. The Central Processing System 103 performs real-time location determination by applying positioning algorithms to the triangulated signal data, generating coordinate positions for tracked entities within the indoor space. The Central Processing System 103 maintains the global coordinate origin reference that serves as the foundational spatial anchor point for all coordinate calculations and position determinations throughout the system.

The Central Processing System 103 operates through the coordination of hardware and software components configured to execute location processing workflows. The Central Processing System 103 includes at least one computing processor that executes the computational operations required for floor plan processing, coordinate calculation, signal triangulation, and position determination. The memory associated with the Central Processing System 103 stores executable instructions that define the operational logic and algorithmic procedures implemented by the processor. The Central Processing System 103 receives floor plan image files through an import mechanism and processes these files to extract spatial information and dimensional data. The Central Processing System 103 interfaces with a structured datastore that maintains polygon definitions specifying the geometric boundaries of spatial zones and location classifications that categorize these zones according to their designated functions or characteristics. The Central Processing System 103 receives signal data from networked ultra-wide band beacons through communication pathways and applies position algorithms to this signal data to calculate the locations of tracked devices or assets. The position algorithms executed by the Central Processing System 103 utilize the signal characteristics from multiple beacons to triangulate positions relative to the established global coordinate origin reference.

Alternative implementations of the Central Processing System 103 may utilize distributed computing architectures wherein processing tasks are allocated across multiple interconnected computing nodes rather than consolidated within a single server unit. The Central Processing System 103 may alternatively be implemented using cloud-based computing resources that provide scalable processing capacity through virtualized infrastructure. The computing processor within the Central Processing System 103 may comprise a general purpose central processing unit, a graphics processing unit configured for parallel computational tasks, a field programmable gate array, or an application specific integrated circuit designed for location processing operations. The memory storing executable instructions may be implemented using volatile random access memory, non-volatile solid state storage, or combinations thereof arranged in hierarchical caching configurations. The structured datastore may be implemented as a relational database management system, a document-oriented database, a graph database optimized for spatial relationships, or an in-memory data structure maintained within the server memory allocation. The position algorithms may implement time difference of arrival calculations, time of arrival measurements, angle of arrival determinations, received signal strength indicator analysis, or hybrid approaches combining multiple signal characteristics. The floor plan data processing may utilize optical character recognition, computer vision edge detection, vector graphic parsing, or manual coordinate entry interfaces. The beacon signal triangulation may employ multilateration techniques, fingerprinting methods based on signal pattern matching, or probabilistic positioning models that account for signal propagation uncertainties. The communication pathways between the Central Processing System 103 and the networked ultra-wide band beacons may utilize wired ethernet connections, wireless local area network protocols, or dedicated radio frequency communication channels.

At a high level the Beacon(s) 120 comprises one or more beacons for communicating with wearables to determine wearable location within a facility. Beacon(s) 120 may comprise an Ultra-Wide Band Beacon Array comprising a distributed network of positioning beacons strategically deployed throughout a facility to enable precise location determination of wearable user devices within the monitored environment. The Beacon(s) 120 operates in conjunction with the Central Processing System 103 and communicates with wearable devices worn by users to establish real-time positional awareness throughout the facility coverage area.

The Beacon(s) 120 provide(s) spatial positioning functionality by transmitting timing signals that enable triangulation-based location calculations for devices operating within range of multiple beacons. The Beacon(s) 120 receives signals from wearable user devices and relays the received signal data to the Central Processing System 103 for processing and location computation. The Beacon(s) 120 achieve(s) positional accuracy to within approximately several feet or even a few inches, thereby enabling the system to determine user locations with sufficient precision for facility management, safety monitoring, and access control applications.

The Beacon(s) 120 operates through a plurality of individual UWB beacons installed at designated locations throughout the facility according to placement coordinates generated by the system. Each beacon within the Beacon(s) 120 include a wireless transceiver configured to communicate with wearable user devices using ultra-wide band radio frequency signals. The transceivers transmit and receive communications at periodic time intervals during normal operation and additionally upon receiving command signals from the Central Processing System 103 or from the wearable devices themselves. The timing signals transmitted by the beacons enable time-of-arrival and time-difference-of-arrival measurements that form the basis for triangulation calculations performed by the Central Processing System 103. When a wearable device transmits a signal, one or more beacons within the Beacon(s) 120 receive the transmission and relay the received signal data along with timestamp information to the Central Processing System 103, which processes the timing differentials to compute device position coordinates.

Alternative implementations of the Beacon(s) 120 may utilize Bluetooth Low Energy beacons configured for received signal strength indication based positioning rather than ultra-wide band timing signals. The Beacon(s) 120 may alternatively comprise WiFi access points configured to perform fine timing measurement protocols for position determination. Radio frequency identification readers operating in active or passive modes may substitute for ultra-wide band beacons in certain implementations. Infrared beacon arrays positioned throughout the facility may provide line-of-sight positioning capabilities as an alternative to radio frequency based approaches. Ultrasonic transducer arrays may replace the ultra-wide band beacons to enable acoustic-based triangulation positioning. The Beacon(s) 120 may alternatively implement chirp spread spectrum technology or frequency modulated continuous wave radar techniques for distance measurement and position determination. Hybrid implementations combining multiple positioning technologies such as ultra-wide band with Bluetooth Low Energy or WiFi fingerprinting may be employed within the beacon array architecture.

At a high level the Wearable(s) 130, also referred to herein as Wearable User Device, generally comprises a portable electronic apparatus configured to be worn on a user’s body and designed to facilitate indoor positioning and tracking within a facility environment. The Wearable User Device 130 operates as a mobile node within the positioning system architecture, interfacing with the beacon array to enable continuous location determination of personnel throughout the monitored premises. The Wearable User Device 130 may be or comprise a user device of the same type as those described in association with user device(s) 110.

The Wearable User Device 130 performs multiple functions within the positioning system. The Wearable User Device 130 transmits identification signals that contain unique device identifiers enabling the system to distinguish between multiple users operating within the same facility space. The Wearable User Device 130 responds to interrogation commands from the beacon array, facilitating time-of-flight measurements that enable precise distance calculations between the device and multiple beacon nodes. The Wearable User Device 130 collects and transmits elevation data to support three-dimensional positioning calculations, particularly in facility areas spanning multiple floor levels. The Wearable User Device 130 maintains periodic communication with the positioning infrastructure to ensure continuous tracking capability even when the user is stationary.

The Wearable User Device 130 operates through an ultra-wide band transceiver module that exchanges radio frequency signals with the beacon array distributed throughout the facility. The ultra-wide band transceiver within the Wearable User Device 130 transmits identification signals at configurable periodic intervals, with these signals propagating to multiple beacons that receive and timestamp the transmissions for position calculation purposes. When the positioning system initiates a real-time ping command, the Wearable User Device 130 receives the command through its ultra-wide band transceiver and generates a response signal that enables the system to perform time-difference-of-arrival or time-of-flight measurements. The Wearable User Device 130 incorporates elevation sensing components that detect vertical position changes, with this elevation data being incorporated into the transmitted signals to enable the positioning engine to resolve three-dimensional coordinates relative to the floor plan global origin established within the system.

Alternative implementations of the Wearable User Device 130 may include smart bands, smart rings, smart pendants, smart badges, or smart clothing items incorporating the positioning transceiver and sensor components. The ultra-wide band transceiver within the Wearable User Device 130 may alternatively comprise Bluetooth Low Energy transceivers, WiFi transceivers operating in fine timing measurement modes, or infrared transceivers. The elevation sensing components within the Wearable User Device 130 may alternatively comprise barometric pressure sensors, accelerometer-based step detection systems with floor change algorithms, or magnetometer arrays configured to detect floor-specific magnetic signatures. The periodic transmission intervals may alternatively be event-triggered based on motion detection, user-initiated through manual activation, or dynamically adjusted based on battery conservation requirements. The identification signals transmitted by the Wearable User Device 130 may alternatively employ encrypted identifiers, rotating pseudonymous identifiers, or challenge-response authentication protocols.

At a high level 140 comprises an administrative client terminal that provides a networked computing device configured to facilitate floor plan configuration operations and real-time monitoring functions through a graphical user interface. The administrative client terminal 140 serves as the primary interface through which system administrators and facility managers interact with the spatial tracking infrastructure, enabling both the initial setup of monitored environments and the ongoing observation of wearable device positions within those environments.

The administrative client terminal 140 performs several interrelated functions within the overall system architecture. The administrative client terminal 140 renders a floor plan canvas that displays graphical representations of physical spaces, including polygon overlays that define discrete location zones and beacon location indicators that show the placement of positioning infrastructure components. The administrative client terminal 140 accepts user input for creating and modifying polygon boundaries through drawing interactions, enables assignment of location categories to defined zones through drag-and-drop interface mechanisms, and presents real-time visual indicators that depict the current positions of wearable devices overlaid upon the floor plan representation. The administrative client terminal 140 communicates with the backend server infrastructure to retrieve floor plan data, submit configuration changes, and receive streaming position updates for display purposes.

The administrative client terminal 140 operates through a combination of display rendering, input processing, and network communication subsystems. The display subsystem of the administrative client terminal 140 renders the floor plan canvas by retrieving stored floor plan imagery and overlaying vector-based polygon shapes that correspond to defined location zones. The administrative client terminal 140 positions beacon indicators at coordinates corresponding to physical beacon installations and updates wearable device position indicators based on location data received from the backend infrastructure. The input processing subsystem of the administrative client terminal 140 captures mouse, touch, or stylus interactions to enable polygon vertex placement during zone definition operations, detects drag-and-drop gestures for associating location category designations with polygon regions, and processes selection interactions for editing existing configuration elements. The network communication subsystem of the administrative client terminal 140 establishes connections with backend server components to synchronize configuration data and receive real-time position streams through persistent connection mechanisms.

Alternative implementations of the administrative client terminal 140 may utilize different computing device form factors including desktop workstations, laptop computers, tablet devices, or dedicated terminal hardware. The graphical user interface of the administrative client terminal 140 may alternatively be implemented as a native application compiled for specific operating system platforms, a web-based application rendered within browser software, or a hybrid application combining native and web-based rendering approaches. The floor plan rendering functionality of the administrative client terminal 140 may alternatively employ raster-based graphics processing, vector graphics rendering libraries, three-dimensional visualization engines, or combinations thereof. The polygon drawing interface of the administrative client terminal 140 may alternatively accept input through coordinate entry fields, automated boundary detection from floor plan imagery, import of polygon definitions from external design software, or voice-guided drawing commands. The drag-and-drop interaction mechanism of the administrative client terminal 140 may alternatively be implemented through context menu selections, keyboard shortcut assignments, touch gesture recognizers, or programmatic category assignment through scripting interfaces. The real-time position display functionality of the administrative client terminal 140 may alternatively receive position data through polling requests, server-sent event streams, message queue subscriptions, or peer-to-peer communication channels. The display peripherals associated with the administrative client terminal 140 may alternatively comprise single monitors, multi-monitor configurations, projection display systems, or head-mounted display devices. The input peripherals associated with the administrative client terminal 140 may alternatively include mouse devices, trackpads, touchscreen interfaces, stylus input devices, gesture recognition sensors, or voice input systems.

At a high level 105 comprises a location data repository implemented as a networked database system that provides centralized storage and management of spatial data, device information, and temporal position records to support indoor positioning and location tracking operations. The location data repository 105 operates as a persistent storage infrastructure that maintains multiple categories of interrelated data including floor plan image data, polygon coordinate definitions, beacon placement coordinates, location category associations, device identification records, and historical location data. The location data repository 105 interfaces with other system components to receive, store, organize, and retrieve location-related information that enables real-time positioning, historical analysis, and reporting functions.

The location data repository 105 stores floor plan image data representing the physical layout of monitored environments, where such image data may include raster images, vector graphics, or hybrid representations of building floors, wings, or facilities. The location data repository 105 maintains polygon coordinate definitions that correspond to discrete spatial regions within the floor plan representations, where each polygon defines the boundaries of a particular zone, room, hallway, or other designated area using coordinate pairs that map to the floor plan coordinate system. The location data repository 105 stores beacon placement coordinates that specify the physical installation positions of wireless beacons throughout the monitored environment, enabling the system to correlate detected beacon signals with known spatial locations. The location data repository 105 maintains location category associations that link polygon-defined regions with classification labels such as zone designations, room identifiers, hallway names, or functional area descriptions.

The location data repository 105 stores device identification records that associate unique device identifiers with corresponding device attributes, user assignments, or asset classifications. The location data repository 105 maintains historical location data comprising time-stamped position records that document the temporal sequence of device locations within the monitored environment. Each time-stamped position record stored in the location data repository 105 includes device coordinates specifying the calculated position of a tracked device, associated beacon identifiers indicating which beacons contributed to the position determination, and corresponding zone, room, and hallway classifications derived from the polygon coordinate definitions and location category associations.

The location data repository 105 operates by receiving position data from location processing components and persisting such data in structured storage formats that enable efficient retrieval and analysis. The location data repository 105 indexes the stored records according to multiple dimensions including device identifier, timestamp, zone classification, and beacon association to support various query patterns. The location data repository 105 provides data retrieval interfaces that enable other system components to access current and historical position information for individual devices, groups of devices, or all devices within specified spatial or temporal boundaries.

The location data repository 105 enables historical movement tracking by maintaining the chronological sequence of position records for each tracked device, allowing reconstruction of device trajectories over specified time periods. The location data repository 105 supports headcount generation by providing query capabilities that return the set of devices located within specified zones or regions at particular points in time or across time ranges. The location data repository 105 facilitates compliance auditing by preserving immutable records of device locations with associated timestamps, enabling verification of presence or absence within designated areas during specified periods.

The location data repository 105 may be implemented using relational database management systems such as MySQL, PostgreSQL, Microsoft SQL Server, or Oracle Database. The location data repository 105 may alternatively be implemented using NoSQL database systems such as MongoDB, Cassandra, or DynamoDB. The location data repository 105 may be implemented using time-series database systems such as InfluxDB, TimescaleDB, or OpenTSDB that provide optimized storage and retrieval for temporal data. The location data repository 105 may utilize distributed database architectures that partition data across multiple storage nodes. The location data repository 105 may employ in-memory database systems such as Redis or Memcached for caching frequently accessed data. The location data repository 105 may be deployed on cloud-based database services such as Amazon RDS, Google Cloud SQL, or Azure SQL Database. The location data repository 105 may utilize graph database systems such as Neo4j or Amazon Neptune for storing and querying spatial relationships. The location data repository 105 may implement spatial database extensions such as PostGIS or Oracle Spatial for optimized geometric data storage and spatial query operations.

User device(s) 110 include, generally, a computer or computing device including functionality for communicating (e.g., remotely) over a network 150. Data may be collected from user devices 110, and data requests may be initiated from each user device 110. User device(s) 110 may be a server, a desktop computer, a laptop computer, personal digital assistant (PDA), an in- or out-of-car navigation system, a smart phone or other cellular or mobile phone, or mobile gaming device, among other suitable computing devices. User devices 110 may execute one or more applications, such as a web browser (e.g., Microsoft Windows Internet Explorer, Mozilla Firefox, Apple Safari, Google Chrome, and Opera, etc.), or a dedicated application to submit user data, or to make prediction queries over a network 150.

In particular embodiments, each user device 110 may be an electronic device including hardware, software, or embedded logic components or a combination of two or more such components and capable of carrying out the appropriate functions implemented or supported by the user device 110. For example and without limitation, a user device 110 may be a desktop computer system, a notebook computer system, a netbook computer system, a handheld electronic device, or a mobile telephone. The present disclosure contemplates any user device 110. A user device 110 may enable a network user at the user device 110 to access network 150. A user device 110 may enable its user to communicate with other users at other user devices 110.

A user device 110 may have a web browser, such as MICROSOFT INTERNET EXPLORER, GOOGLE CHROME or MOZILLA FIREFOX, and may have one or more add-ons, plug-ins, or other extensions, such as TOOLBAR or YAHOO TOOLBAR. A user device 110 may enable a user to enter a Uniform Resource Locator (URL) or other address directing the web browser to a server, and the web browser may generate a Hyper Text Transfer Protocol (HTTP) request and communicate the HTTP request to server. The server may accept the HTTP request and communicate to the user device 110 one or more Hyper Text Markup Language (HTML) files responsive to the HTTP request. The user device 110 may render a web page based on the HTML files from server for presentation to the user. The present disclosure contemplates any suitable web page files. As an example and not by way of limitation, web pages may render from HTML files, Extensible Hyper Text Markup Language (XHTML) files, or Extensible Markup Language (XML) files, according to particular needs. Such pages may also execute scripts such as, for example and without limitation, those written in JAVASCRIPT, JAVA, MICROSOFT SILVERLIGHT, combinations of markup language and scripts such as AJAX (Asynchronous JAVASCRIPT and XML), and the like. Herein, reference to a web page encompasses one or more corresponding web page files (which a browser may use to render the web page) and vice versa, where appropriate.

The user device 110 may also include an application that is loaded onto the user device 110. The application obtains data from the network 150 and displays it to the user within the application interface.

Exemplary user devices are illustrated in some of the subsequent figures provided herein. This disclosure contemplates any suitable number of user devices, including computing systems taking any suitable physical form. As example and not by way of limitation, computing systems may be an embedded computer system, a system-on-chip (SOC), a single-board computer system (SBC) (such as, for example, a computer-on-module (COM) or system-on-module (SOM)), a desktop computer system, a laptop or notebook computer system, an interactive kiosk, a mainframe, a mesh of computer systems, a mobile telephone, a personal digital assistant (PDA), a server, or a combination of two or more of these. Where appropriate, the computing system may include one or more computer systems; be unitary or distributed; span multiple locations; span multiple machines; or reside in a cloud, which may include one or more cloud components in one or more networks. Where appropriate, one or more computing systems may perform without substantial spatial or temporal limitation one or more steps of one or more methods described or illustrated herein. As an example, and not by way of limitation, one or more computing systems may perform in real time or in batch mode one or more steps of one or more methods described or illustrated herein. One or more computing system may perform at different times or at different locations one or more steps of one or more methods described or illustrated herein, where appropriate.

Network cloud 150 generally represents a network or collection of networks (such as the Internet or a corporate intranet, or a combination of both) over which the various components illustrated in FIG. 1 (including other components that may be necessary to execute the system described herein, as would be readily understood to a person of ordinary skill in the art). In particular embodiments, network 150 is an intranet, an extranet, a virtual private network (VPN), a local area network (LAN), a wireless LAN (WLAN), a wide area network (WAN), a metropolitan area network (MAN), a portion of the Internet, or another network 150 or a combination of two or more such networks 150. One or more links connect the systems and databases described herein to the network 150. In particular embodiments, one or more links each includes one or more wired, wireless, or optical links. In particular embodiments, one or more links each includes an intranet, an extranet, a VPN, a LAN, a WLAN, a WAN, a MAN, a portion of the Internet, or another link or a combination of two or more such links. The present disclosure contemplates any suitable network 150, and any suitable link for connecting the various systems and databases described herein.

The network 150 connects the various systems and computing devices described or referenced herein. In particular embodiments, network 150 is an intranet, an extranet, a virtual private network (VPN), a local area network (LAN), a wireless LAN (WLAN), a wide area network (WAN), a metropolitan area network (MAN), a portion of the Internet, or another network 421 or a combination of two or more such networks 150. The present disclosure contemplates any suitable network 150.

One or more links couple one or more systems, engines or devices to the network 150. In particular embodiments, one or more links each includes one or more wired, wireless, or optical links. In particular embodiments, one or more links each includes an intranet, an extranet, a VPN, a LAN, a WLAN, a WAN, a MAN, a portion of the Internet, or another link or a combination of two or more such links. The present disclosure contemplates any suitable links coupling one or more systems, engines or devices to the network 150.

In particular embodiments, each system or engine may be a unitary server or may be a distributed server spanning multiple computers or multiple datacenters. Systems, engines, or modules may be of various types, such as, for example and without limitation, web server, news server, mail server, message server, advertising server, file server, application server, exchange server, database server, or proxy server. In particular embodiments, each system, engine or module may include hardware, software, or embedded logic components or a combination of two or more such components for carrying out the appropriate functionalities implemented or supported by their respective servers. For example, a web server is generally capable of hosting websites containing web pages or particular elements of web pages. More specifically, a web server may host HTML files or other file types, or may dynamically create or constitute files upon a request, and communicate them to client/user devices or other devices in response to HTTP or other requests from client devices or other devices. A mail server is generally capable of providing electronic mail services to various client devices or other devices. A database server is generally capable of providing an interface for managing data stored in one or more data stores.

In particular embodiments, one or more data storages may be communicatively linked to one or more servers via one or more links. In particular embodiments, data storages may be used to store various types of information. In particular embodiments, the information stored in data storages may be organized according to specific data structures. In particular embodiments, each data storage may be a relational database. Particular embodiments may provide interfaces that enable servers or clients to manage, e.g., retrieve, modify, add, or delete, the information stored in data storage.

The system may also contain other subsystems and databases, which are not illustrated in FIG. 1, but would be readily apparent to a person of ordinary skill in the art. For example, the system may include databases for storing data, storing features, storing outcomes (training sets), and storing models. Other databases and systems may be added or subtracted, as would be readily understood by a person of ordinary skill in the art, without departing from the scope of the invention.

### System Architecture

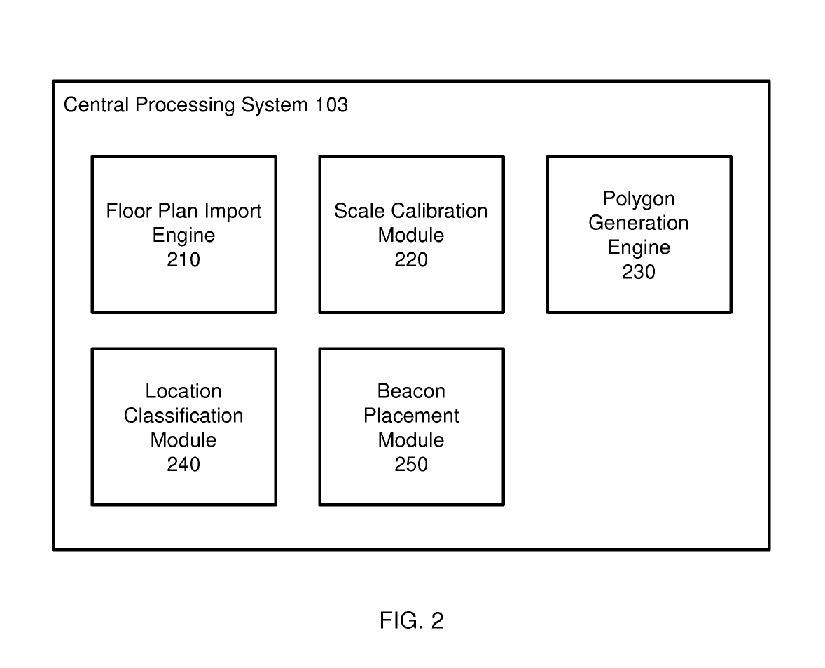

FIG. 2 illustrates an exemplary embodiment of the Floor Plan Mapping System for Real-Time Device Location Tracking. In particular, FIG. 2 illustrates an exemplary embodiment of the Central Processing System 103 comprised of Floor Plan Import Engine 210, Scale Calibration Module 220, Polygon Generation Engine 230, Location Classification Module 240, and Beacon Placement Module 250. The various components described herein are exemplary and for illustration purposes only and any combination or subcombination of the various components may be used as would be apparent to one of ordinary skill in the art. Other systems, interfaces, modules, engines, databases, and the like, may be used, as would be readily understood by a person of ordinary skill in the art, without departing from the scope of the invention. Any system, interface, module, engine, database, and the like may be divided into a plurality of such elements for achieving the same function without departing from the scope of the invention. Any system, interface, module, engine, database, and the like may be combined or consolidated into fewer of such elements for achieving the same function without departing from the scope of the invention. All functions of the components discussed herein may be initiated manually or may be automatically initiated when the criteria necessary to trigger action have been met.

At a high level, Floor Plan Import Engine 210 comprises a software subsystem configured to receive, process, and prepare facility floor plan image data for subsequent utilization within the broader system architecture. The Floor Plan Import Engine 210 serves as an ingestion mechanism that transforms raw image files depicting architectural layouts into optimized digital representations suitable for interactive manipulation and spatial computation operations.

The Floor Plan Import Engine 210 performs several interconnected functions within the system. A first aspect of the invention involves the reception and parsing of imported image files containing facility floor plan data in various digital formats. The subsystem 210 accepts image data that may be encoded in formats including, but not limited to, JPEG, PNG, TIFF, BMP, PDF, or vector-based formats such as SVG or DXF. Upon receipt of the image file, the Floor Plan Import Engine 210 extracts metadata associated with the file, which may include dimensional information, resolution parameters, color depth specifications, and embedded coordinate data when available.

A further aspect of the Floor Plan Import Engine 210 involves the execution of image optimization algorithms designed to reduce the storage footprint of the imported floor plan data while preserving visual fidelity sufficient for user interaction and spatial analysis. The optimization processing may encompass lossy or lossless compression techniques, resolution scaling operations, color palette reduction, and format conversion procedures. The optimization algorithms implemented within subsystem 210 may utilize adaptive compression ratios determined based on the complexity of the source image, the intended display resolution, and system memory constraints.

The Floor Plan Import Engine 210 additionally performs loading operations whereby the optimized image data is transferred into a canvas rendering buffer. The canvas rendering buffer comprises a memory allocation structure configured to store pixel data in a format amenable to rapid display rendering and interactive manipulation. The subsystem 210 manages memory allocation for the canvas rendering buffer and establishes data structures that facilitate efficient access to image pixel data during subsequent rendering operations.

An aspect of the Floor Plan Import Engine 210 involves the establishment of a global coordinate origin positioned at the absolute top-left corner of the imported floor plan image. This coordinate origin serves as the reference point from which all spatial calculations within the system are performed. The subsystem 210 initializes a coordinate system wherein the horizontal axis extends rightward from the origin with increasing positive values, and the vertical axis extends downward from the origin with increasing positive values. Spatial positions within the floor plan are thereafter expressed as coordinate pairs relative to this established origin point.

The operational methodology of the Floor Plan Import Engine 210 may be understood through examination of the processing pipeline. Upon initiation of an import operation, the subsystem 210 first validates the incoming file to confirm compatibility with supported format specifications. File validation may include verification of file header information, assessment of file integrity through checksum validation, and confirmation that the file size falls within acceptable parameters. Following successful validation, the subsystem 210 instantiates a decoder module corresponding to the detected file format, which parses the binary file data and extracts raw image information.

The extracted image data is subsequently passed to an optimization module within the Floor Plan Import Engine 210. The optimization module analyzes characteristics of the source image including dimensions, color distribution, and edge complexity. Based on this analysis, the optimization module selects and applies appropriate compression and scaling algorithms. The optimization process may involve iterative refinement wherein multiple compression levels are evaluated to identify a configuration that achieves target file size reduction while maintaining image quality above a defined threshold.

Following optimization, the Floor Plan Import Engine 210 transfers the processed image data to the canvas rendering buffer. This transfer operation may involve format conversion to ensure compatibility with the rendering subsystem’s expected data structure. The subsystem 210 establishes pointers or references to the buffer location and populates associated data structures with dimensional parameters and coordinate mapping information.

Alternative implementations of the Floor Plan Import Engine 210 may be employed without departing from the scope of the invention. In one alternative embodiment, the subsystem 210 may utilize cloud-based processing resources to perform computationally intensive optimization operations, thereby reducing local processing requirements. In another alternative embodiment, the Floor Plan Import Engine 210 may implement a streaming architecture wherein large floor plan images are processed and loaded in segmented tiles rather than as a unified image, facilitating handling of high-resolution source files. A further alternative involves the utilization of hardware-accelerated image processing through graphics processing unit resources to expedite optimization and rendering buffer loading operations. The coordinate origin may alternatively be established at other positions within the image bounds, with appropriate transformation matrices applied to maintain consistency with downstream spatial calculation requirements.

At a high level, Scale Calibration Module 220 comprises a software component within the system architecture that establishes dimensional relationships between pixel-based measurements in a digital floor plan representation and corresponding real-world distance values. The Scale Calibration Module 220 functions as an intermediary processing layer that transforms abstract pixel coordinates into meaningful physical measurements, thereby enabling accurate spatial calculations throughout the floor plan analysis system.

The Scale Calibration Module 220 performs several interconnected functions within the broader system framework. A primary function of 220 involves receiving coordinate input data representing two distinct points selected within a digital floor plan image or representation. These first and second coordinate inputs correspond to endpoints of a known dimension within the depicted space, such as a wall segment, room length, doorway width, or other architectural feature having a determinable real-world measurement. The module 220 additionally accepts user-specified or system-provided real-world dimension values that correspond to the actual physical distance between the selected coordinate points. Upon receiving these inputs, the Scale Calibration Module 220 generates a pixel-to-distance conversion ratio that serves as a scaling factor for subsequent measurement operations performed by other components within the system.

In terms of operational methodology, the Scale Calibration Module 220 implements a coordinate processing pipeline that begins with the acquisition of first coordinate data comprising x and y pixel position values and second coordinate data similarly comprising x and y pixel position values. The module 220 calculates the Euclidean distance between these two coordinate points using standard geometric computation techniques, yielding a pixel-based distance measurement. The Scale Calibration Module 220 then divides the user-specified real-world dimension value by the calculated pixel distance to derive the pixel-to-distance conversion ratio. This ratio represents the real-world distance equivalent of a single pixel unit within the floor plan representation.

An aspect of the Scale Calibration Module 220 involves the enforcement of aspect ratio constraints during the calibration process. The module 220 maintains proportional accuracy by analyzing the relationship between horizontal and vertical scaling factors to detect and compensate for potential distortions in the source floor plan image. In embodiments where the source image exhibits non-uniform scaling characteristics, the Scale Calibration Module 220 may apply correction factors or generate separate horizontal and vertical conversion ratios to preserve measurement accuracy across different dimensional axes. The aspect ratio constraint enforcement mechanism within 220 may compare calculated ratios against predetermined tolerance thresholds and generate notification outputs when detected aspect ratio deviations exceed acceptable parameters.

The Scale Calibration Module 220 may store the calculated conversion ratio and associated calibration metadata in a data structure accessible to other system components. This calibration data may include timestamp information, source coordinate values, the specified real-world dimension, confidence metrics, and aspect ratio validation results. The module 220 may implement caching mechanisms to retain calibration data for subsequent measurement operations without requiring recalculation.

Alternative implementations of the Scale Calibration Module 220 may utilize different approaches for establishing pixel-to-distance relationships. In one alternative embodiment, the module 220 may accept multiple coordinate pairs along multiple known dimensions and calculate an averaged or weighted conversion ratio to improve calibration accuracy. Another alternative implementation may incorporate automatic dimension detection capabilities wherein the module 220 identifies standard architectural elements such as doors or windows having known typical dimensions and derives conversion ratios without explicit user input of real-world measurements. The Scale Calibration Module 220 may alternatively implement machine learning models trained on architectural datasets to estimate scaling factors based on recognized floor plan features and patterns.

Further alternative embodiments of 220 may utilize reference markers or fiducial indicators embedded within floor plan images to establish scale calibration automatically. The module 220 may alternatively accept scale bar inputs commonly found in architectural drawings and extract conversion ratios through image analysis of the scale bar representation. In yet another alternative, the Scale Calibration Module 220 may interface with external data sources containing building information or property records to obtain reference dimensions for calibration purposes. The module 220 may also implement iterative calibration refinement processes wherein initial conversion ratios are adjusted based on cross-validation against multiple known dimensions within the floor plan representation.

At a high level, Polygon Generation Engine 230 comprises a software subsystem configured to transform user interface interactions into structured geometric data representations that define discrete location areas within a floor plan environment. The Polygon Generation Engine 230 serves as an intermediary processing layer between user input mechanisms and the underlying location data storage infrastructure, facilitating the creation of precise spatial boundaries for various architectural and functional zones.

The Polygon Generation Engine 230 performs several interrelated functions within the system architecture. An aspect of the invention involves receiving coordinate input data from user interface components, wherein such input data may be derived from mouse clicks, touch gestures, stylus interactions, or programmatic input streams. The subsystem 230 processes these raw input signals to derive vertex positions that collectively define polygon boundaries. The Polygon Generation Engine 230 generates polygon coordinate sets that correspond to multiple location classification types, including but not limited to zones, floors, rooms, hallways, and exterior locations. Each polygon generated by subsystem 230 represents a closed geometric shape that demarcates a specific area within the floor plan coordinate space.

The operational methodology of the Polygon Generation Engine 230 involves several processing stages. Upon receiving user interface inputs, the subsystem 230 performs coordinate transformation operations to map screen-relative positions to floor plan coordinates referenced against a global origin point established within the system. The global origin serves as a reference datum from which all vertex coordinates are calculated, thereby maintaining spatial consistency across multiple polygon definitions. The Polygon Generation Engine 230 implements vertex assignment algorithms that sequentially process input points and assign coordinate values expressed in relation to the global origin coordinate system.

A part of the polygon generation process implemented by subsystem 230 includes validation routines that verify polygon closure, wherein the engine confirms that the final vertex connects to the initial vertex to form a complete bounded region. The subsystem 230 may implement geometric processing functions such as vertex ordering algorithms to maintain consistent winding direction, intersection detection to identify self-intersecting polygon configurations, and area calculation routines to quantify the spatial extent of generated polygons.

The Polygon Generation Engine 230 interfaces with the location data repository to persist polygon definitions following generation. The storage operations performed by subsystem 230 may include serialization of coordinate arrays, association of polygon geometries with location classification metadata, and indexing operations to facilitate subsequent retrieval and spatial query operations.

An aspect of the Polygon Generation Engine 230 encompasses support for various polygon complexity levels, ranging from simple convex quadrilaterals to complex concave polygons with numerous vertices. The subsystem 230 may implement vertex simplification algorithms to reduce polygon complexity while maintaining geometric fidelity within specified tolerance parameters. Additionally, the Polygon Generation Engine 230 may support polygon modification operations including vertex insertion, vertex deletion, vertex repositioning, and polygon subdivision.

Alternative implementations of the Polygon Generation Engine 230 may utilize different geometric representation schemes. In one alternative embodiment, subsystem 230 may generate polygon definitions using polar coordinate systems rather than Cartesian coordinate systems, wherein vertices are expressed as radius and angle values relative to a centroid or reference point. Another alternative implementation of 230 may employ parametric curve representations such as Bezier curves or B-splines to define polygon boundaries with smooth curvature characteristics.

The Polygon Generation Engine 230 may alternatively implement grid-snapping functionality wherein vertex coordinates are constrained to discrete grid positions, facilitating alignment with architectural elements and standardized dimensional increments. In another alternative configuration, subsystem 230 may utilize template-based polygon generation wherein predefined geometric shapes are instantiated and transformed according to user-specified parameters including position, scale, and rotation values.

Alternative storage mechanisms for polygon definitions generated by subsystem 230 may include vector graphics formats, geographic information system data structures, or custom binary serialization schemes optimized for spatial indexing and retrieval performance. The Polygon Generation Engine 230 may alternatively implement distributed processing architectures wherein polygon generation computations are partitioned across multiple processing nodes for enhanced throughput in scenarios involving large-scale floor plan environments with numerous location definitions.

At a high level, Location Classification Module 240 provides a classification framework that enables users to assign semantic labels and categorical designations to polygon geometries generated within the system. This location classification module 240 facilitates the organization and categorization of spatial regions through an interactive user interface paradigm, thereby enabling the system to distinguish between different functional zones within a monitored environment.

In terms of functionality, location classification module 240 receives polygon data structures from upstream processing components and presents these geometries within a graphical user interface configured to accept drag-and-drop interactions. The module 240 maintains a taxonomy of predefined classification categories including, but not limited to, housing areas, hallways, restricted zones, and keep-away areas. When a user initiates a drag-and-drop operation, location classification module 240 captures the interaction event, identifies the target polygon, and associates the selected classification label with that polygon’s data record. The module 240 further processes classifications related to device presence constraints, wherein certain areas may be designated as zones where devices should not be present or where limited access requirements apply. These device-related classifications enable downstream components to enforce spatial constraints on tracked objects or personnel.

Regarding operational mechanics, location classification module 240 implements an event-driven architecture wherein user interface interactions trigger classification assignment procedures. The module 240 instantiates a classification handler that monitors drag-and-drop events within the rendering canvas. Upon detection of a valid drop operation over a polygon region, the handler extracts the classification identifier from the dragged element and invokes a data association routine. This routine generates a classification record comprising the polygon identifier, the classification category identifier, timestamp metadata, and any auxiliary parameters such as access restriction levels or device exclusion flags. Location classification module 240 then transmits this classification record to the datastore component for persistent storage, wherein the record is indexed by both polygon identifier and classification category to facilitate efficient retrieval operations. The module 240 may implement validation logic to verify that classification assignments conform to predefined rules, such as preventing conflicting classifications on a single polygon or enforcing hierarchical classification constraints.

An aspect of the invention is comprised of the user interface component of location classification module 240 rendering visual indicators that communicate classification status to the operator. These indicators may include color-coded overlays, pattern fills, iconographic markers, or textual labels applied to classified polygons. The module 240 maintains a bidirectional synchronization mechanism with the datastore such that classification modifications performed through the interface are reflected in persistent storage and vice versa.

Alternative embodiments of location classification module 240 may employ different interaction paradigms for classification assignment. In one alternative, the module 240 may implement a context menu interface wherein users right-click on polygon regions to access a classification selection menu. Another alternative involves a toolbar-based approach wherein users select a classification tool and subsequently click on target polygons to apply the selected classification. A further alternative embodiment utilizes automated classification through machine learning algorithms that analyze polygon characteristics such as shape, size, position, and proximity to other classified regions to suggest or automatically assign classifications. In yet another alternative, location classification module 240 may implement a form-based interface wherein users select polygons and complete classification forms with dropdown menus, checkbox controls, or text input fields. The datastore interaction component of location classification module 240 may alternatively utilize different persistence mechanisms including relational database systems, document-oriented databases, graph databases, or distributed key-value stores. The classification taxonomy maintained by location classification module 240 may alternatively be implemented as a configurable schema that administrators can modify to add custom classification categories, or as a hierarchical ontology supporting inheritance relationships between classification types.

At a high level, Beacon Placement Module 250 comprises a computational subsystem configured to determine spatial coordinates for ultra-wide band (UWB) beacon deployment within a facility environment. The beacon placement module 250 receives floor plan data and generates output coordinate positions referenced to a global origin point, wherein such positions define installation locations for UWB beacon hardware devices. The beacon arrangements produced by beacon placement module 250 facilitate triangulation-based position determination capabilities for wearable tracking devices operating within the facility boundaries.

Regarding functionality, beacon placement module 250 performs geometric analysis of floor plan extents to calculate beacon placement coordinates that provide wireless signal coverage across designated tracking zones. The beacon placement module 250 processes input parameters including floor plan dimensions, structural obstruction data, and coverage requirements to generate beacon location specifications. An aspect of the invention is comprised of beacon placement module 250 outputting coordinate triplets (x, y, z) relative to the established global origin, wherein each coordinate triplet defines a discrete beacon installation position. The beacon placement module 250 further generates placement arrangements that enable position triangulation through geometric relationships between multiple beacon devices and mobile wearable receivers.

With respect to operational methodology, beacon placement module 250 implements coverage optimization algorithms that evaluate potential beacon positions against signal propagation models. A part of the computational process involves beacon placement module 250 analyzing floor plan geometry to identify regions requiring beacon coverage and calculating minimum beacon quantities sufficient for triangulation geometry. The beacon placement module 250 applies constraint satisfaction techniques to ensure that generated beacon positions maintain adequate inter-beacon spacing while avoiding placement conflicts with structural elements. Beacon placement module 250 may incorporate signal attenuation modeling to account for wall materials and other obstructions that affect UWB propagation characteristics. The generator beacon placement module 250 iteratively refines beacon positions through optimization routines that minimize coverage gaps while balancing deployment costs associated with beacon quantity.

Alternative implementations of beacon placement module 250 may utilize grid-based placement methodologies wherein beacon coordinates conform to regular spatial intervals across the floor plan extent. In another embodiment, beacon placement module 250 may employ machine learning models trained on historical deployment configurations to predict beacon placements. Beacon placement module 250 may alternatively implement genetic algorithm approaches for multi-objective optimization balancing coverage completeness against beacon count minimization. A further alternative comprises beacon placement module 250 utilizing ray-tracing simulation techniques to model signal propagation and validate coverage prior to finalizing beacon coordinates. Beacon placement module 250 may also implement manual override capabilities permitting user-specified beacon positions that supplement algorithmically generated coordinates.

### Computer Implemented Process

The process steps described herein may be performed in association with a system such as that described in FIG. 1 and/or FIG. 2 above or in association with a different system. The process includes: Obtain Floor Plan Data 310, Define Global Origin 320, Calibrate Floor Plan Scale 330, Generate Location Polygons 340, Assign Location Classifications 350, Generate Beacon Placement Coordinates 360, and Render Floor Plan Display with Overlays 370. The process may comprise additional steps, fewer steps, and/or a different order of steps without departing from the scope of the invention as would be apparent to one of ordinary skill in the art.

At 310, the processor obtains floor plan data by, for example, importing an image file associated with a facility floor plan, wherein the floor plan indicates at least one of a zone, floor, room, mezzanine, hallway, and exterior location, executes image optimization to reduce storage size, and loads the image into a canvas rendering buffer in memory.

At 310, the system acquires spatial representation data corresponding to a physical facility layout through an image file importation process. The facility floor plan image file encompasses graphical representations of architectural elements including, but not limited to, zones defining functional areas within the facility, individual floors in multi-story structures, discrete rooms or enclosed spaces, mezzanine levels representing intermediate floor structures, hallways serving as circulation pathways, and exterior locations adjacent to or surrounding the facility structure. The floor plan data serves as a foundational spatial reference upon which subsequent processing operations and data overlays may be applied.

At 310, the processor interfaces with a file system or network storage location to retrieve the image file containing the floor plan data. The image file may be encoded in various raster formats including PNG, JPEG, BMP, TIFF, or GIF, or alternatively in vector formats such as SVG or PDF. Upon successful retrieval of the image file, the processor parses header information to determine image dimensions, color depth, compression encoding, and metadata attributes associated with the floor plan representation.

At 310, the processor executes image optimization routines to reduce the storage size of the imported floor plan image. The optimization process may employ lossy compression algorithms that selectively discard image data deemed less perceptible to human vision, or lossless compression algorithms that reduce file size through entropy encoding without permanent data loss. The optimization routines may include resolution scaling operations that resample the image to target dimensions appropriate for the intended display context, color palette reduction that decreases the number of distinct color values represented in the image, and removal of embedded metadata or auxiliary data channels not required for rendering purposes.

At 310, following optimization, the processor allocates a canvas rendering buffer within system memory and loads the processed floor plan image into this buffer structure. The canvas rendering buffer comprises a contiguous memory region configured to store pixel data in a format compatible with graphics rendering operations. The buffer may be implemented as a two-dimensional array structure with dimensions corresponding to the image width and height, wherein each array element stores color channel values for the corresponding pixel location. The processor transfers the optimized image data into the allocated buffer through memory copy operations, establishing the floor plan representation in a state ready for subsequent rendering, annotation, or interactive manipulation operations.

At 310, alternative implementations for obtaining floor plan data may include receiving the image file through a network communication interface via HTTP, FTP, or WebSocket protocols from a remote server or cloud storage service. The processor may alternatively obtain floor plan data by capturing the image directly from a connected imaging device such as a scanner or camera. In another alternative, the processor may retrieve floor plan data from a database system wherein the image is stored as a binary large object with associated relational metadata. The floor plan data may alternatively be obtained through integration with computer-aided design systems that export floor plan representations in compatible image formats, or through building information modeling platforms that generate two-dimensional floor plan projections from three-dimensional architectural models.

At 310, alternative implementations for image optimization may include utilizing hardware-accelerated compression through dedicated graphics processing units or specialized image processing coprocessors. The optimization process may alternatively employ adaptive compression techniques that apply varying compression ratios to different regions of the floor plan image based on content complexity analysis. In another alternative, the processor may implement progressive encoding schemes that generate multiple resolution versions of the floor plan image for bandwidth-efficient transmission and incremental rendering capabilities.

At 310, alternative implementations for the canvas rendering buffer may include utilizing graphics processing unit memory through graphics API frameworks such as OpenGL, DirectX, or Vulkan for hardware-accelerated rendering operations. The buffer may alternatively be implemented using off-screen rendering contexts provided by platform-specific windowing systems. In another alternative, the processor may employ double-buffering or triple-buffering techniques wherein multiple canvas buffers are maintained to enable smooth visual transitions during floor plan updates or animations. The canvas rendering buffer may alternatively be implemented as a texture object within a graphics pipeline, enabling efficient compositing with additional graphical layers representing sensor data, annotations, or interactive interface elements overlaid upon the floor plan representation.

At 320, the processor defines a global coordinate origin by assigning a first set of coordinates at the absolute top-left corner of the imported floor plan image. This coordinate origin establishment operation provides a reference framework for all spatial computations performed throughout the system.

At 320, the software process receives the imported floor plan image data and identifies the boundary extents of the image file. The processor examines the image dimensions and locates the uppermost and leftmost pixel position within the image data structure. This corner position is designated as the global coordinate origin, and the processor assigns coordinate values to this position, typically represented as (0, 0) in a two-dimensional Cartesian coordinate system.

At 320, the global coordinate origin functions as an anchor point from which all subsequent coordinate calculations and position determinations are performed. When the system processes spatial data associated with the floor plan, including but not limited to furniture placements, room boundaries, wall segments, doorways, and other architectural elements, the processor calculates the positions of these elements relative to the established global origin point at the top-left corner. This relative positioning approach enables consistent spatial referencing across all operations performed on the floor plan data.

At 320, the processor implements the coordinate system by storing the origin point coordinates in a memory location accessible to subsequent processing modules. The system maintains this origin reference throughout the processing pipeline, ensuring that coordinate transformations, distance calculations, and positional queries all utilize the same reference framework. The top-left corner convention aligns with standard image coordinate conventions where the y-axis increases in the downward direction and the x-axis increases toward the right.

At 320, alternative implementations may utilize different origin positions for the global coordinate system. The processor may alternatively define the global coordinate origin at the center point of the imported floor plan image, which may facilitate certain rotational transformations and symmetric calculations. In another alternative, the processor may establish the global coordinate origin at the bottom-left corner of the floor plan image, conforming to mathematical Cartesian conventions where the y-axis increases in the upward direction. The processor may alternatively define the origin at a user-specified location within or outside the floor plan boundaries, enabling alignment with external coordinate systems or building reference points. In yet another alternative, the processor may establish the origin at a detected architectural feature within the floor plan, such as a corner of a primary room or a structural column location, providing a semantically meaningful reference point for the coordinate system.

At 330, the processor receives first and second coordinate inputs via the user interface corresponding to points along a known dimension, receives a specified real-world dimension value, and calculates a pixel-to-distance conversion ratio while maintaining the aspect ratio unchanged throughout the floor plan.

At 330, the system establishes a calibration mechanism that enables accurate spatial measurement within a digital floor plan representation. The processor is configured to receive user-defined reference points that correspond to a physical dimension of known length within the depicted space. This calibration process transforms the floor plan from a purely visual representation into a metrically accurate document capable of supporting precise dimensional calculations.

At 330, the processor captures two discrete coordinate inputs through the user interface, wherein each coordinate input represents a specific pixel location within the displayed floor plan image. The first coordinate input corresponds to a starting point along a reference dimension, while the second coordinate input corresponds to an ending point along the same reference dimension. These coordinate inputs may be captured through various input mechanisms including, but not limited to, mouse click events, touch screen tap gestures, stylus inputs, or programmatic coordinate designation. The user selects these points along a structural element or feature within the floor plan that has a known real-world measurement, such as a wall segment, doorway width, room length, or other architectural feature whose physical dimension can be verified or is otherwise known to the user.

At 330, concurrent with or subsequent to receiving the coordinate inputs, the processor receives a specified real-world dimension value through the user interface. This dimension value represents the actual physical measurement between the two selected points in real-world units. The user interface may present an input field, dialog box, or other data entry mechanism configured to accept numerical values along with associated unit designations. The real-world dimension value may be expressed in various measurement units including feet, inches, meters, centimeters, or other standard or customary units of linear measurement. The processor may be configured to accept the dimension value in a default unit or may provide unit selection capabilities allowing the user to specify the measurement system employed.

At 330, upon receiving both the coordinate inputs and the specified real-world dimension value, the processor executes a calculation routine to determine the pixel-to-distance conversion ratio. The processor first calculates the pixel distance between the first and second coordinate inputs using geometric distance formulas. In one implementation, the processor applies the Euclidean distance formula, computing the square root of the sum of the squared differences between the x-coordinates and the squared differences between the y-coordinates of the two points. The resulting pixel distance value represents the number of pixels spanning the reference dimension within the digital floor plan image.

At 330, the processor then divides the specified real-world dimension value by the calculated pixel distance to produce the pixel-to-distance conversion ratio. This ratio establishes a scaling factor that defines the real-world distance represented by each pixel in the floor plan image. The conversion ratio may be stored as a floating-point value to preserve precision for subsequent measurement calculations. The processor may store this ratio in association with the floor plan data structure, enabling persistent calibration across user sessions and supporting accurate measurements throughout the floor plan without requiring recalibration.

At 330, the processor maintains the aspect ratio unchanged throughout the floor plan during and after the calibration process. The aspect ratio, defined as the proportional relationship between the horizontal and vertical dimensions of the floor plan image, remains constant to preserve the geometric accuracy of the depicted space. The processor applies the calculated pixel-to-distance conversion ratio uniformly across both horizontal and vertical axes, ensuring that measurements taken in any direction within the floor plan yield proportionally accurate results. This uniform application prevents distortion that would otherwise compromise measurement accuracy and spatial relationships within the floor plan representation.

At 330, alternative implementations for receiving coordinate inputs may include automated point detection wherein the processor employs image recognition algorithms to identify and select reference points based on detected architectural features. The processor may alternatively receive coordinate inputs through keyboard-based numerical entry of pixel coordinates, voice-activated coordinate specification, or imported coordinate data from external measurement devices or CAD systems. The coordinate inputs may also be received through gesture-based interactions on touch-enabled devices, wherein the user draws a line segment connecting the reference points rather than selecting discrete point locations.

At 330, alternative implementations for the distance calculation may employ Manhattan distance calculations for floor plans aligned with orthogonal axes, wherein the processor sums the absolute differences between x-coordinates and y-coordinates. The processor may alternatively implement weighted distance calculations that account for perspective distortion in floor plans captured from photographs or scanned documents. In implementations involving three-dimensional floor plan data, the processor may extend the distance calculation to incorporate z-coordinate differences.

At 330, alternative implementations for the conversion ratio calculation may include multi-point calibration wherein the processor receives multiple pairs of reference points and corresponding dimension values, calculating an averaged or weighted conversion ratio to improve accuracy. The processor may alternatively implement zone-based calibration for floor plans exhibiting non-uniform scaling, wherein different regions of the floor plan maintain separate conversion ratios. The processor may also receive the conversion ratio directly as a user-specified value or extract the ratio from metadata embedded within the floor plan image file.

At 340, the processor assigns a second set of coordinates within the floor plan based on user interface input to generate at least one polygon, wherein each polygon defines a location area associated with at least one tracking beacon and corresponds to a zone, floor, room, hallway, or exterior location of the facility.

At 340, the system receives coordinate data through a user interface mechanism that enables an operator to define spatial boundaries within the previously established floor plan representation. The processor interprets the user interface input as a series of coordinate points that, when connected in sequence, form closed polygonal shapes overlaid upon the floor plan. Each polygon generated through this coordinate assignment process delineates a discrete location area within the facility’s spatial representation.

At 340, the polygon generation process operates by capturing successive coordinate inputs from the user interface, wherein each input corresponds to a vertex position of the polygon being defined. The processor stores these coordinates in memory and mathematically connects adjacent vertices to form line segments that collectively constitute the polygon boundary. Upon receiving an indication that the polygon definition is complete, such as a closing command or selection of the initial vertex, the processor validates the polygon geometry to confirm proper closure and non-self-intersection properties.

At 340, each generated polygon is programmatically associated with at least one tracking beacon positioned within or proximate to the defined location area. This association enables the system to correlate beacon detection signals with specific geographic regions of the facility. The processor maintains a data structure linking each polygon to its corresponding beacon identifier or identifiers, thereby establishing a mapping between physical beacon hardware and logical location areas.

At 340, the polygons correspond to various facility subdivisions including zones, floors, rooms, hallways, or exterior locations. A zone may encompass multiple rooms or represent a functional area spanning several structural divisions. A floor polygon may define the entire boundary of a single level within a multi-story facility. Room polygons delineate individual enclosed spaces such as offices, conference rooms, storage areas, or patient rooms. Hallway polygons define corridor spaces connecting other location areas. Exterior location polygons represent outdoor areas surrounding the facility structure, such as parking areas, loading docks, or campus grounds.

At 340, the user interface input may be provided through various input mechanisms. In one embodiment, the user interface comprises a graphical interface wherein an operator uses a pointing device such as a mouse or trackpad to click on positions within the displayed floor plan, with each click registering a coordinate point. In another embodiment, the user interface accepts touch input on a touchscreen display, wherein the operator touches positions on the floor plan to define polygon vertices. In a further embodiment, the user interface provides text-based coordinate entry fields wherein the operator manually inputs numerical coordinate values. In yet another embodiment, the user interface supports stylus input on a digitizing tablet or touch-enabled display.

At 340, the coordinate system employed for the second set of coordinates may utilize various reference frameworks. In one implementation, the coordinates are expressed as pixel positions relative to the floor plan image origin. In another implementation, the coordinates represent real-world measurements such as feet or meters from a defined reference point within the facility. In a further implementation, the coordinates utilize a normalized coordinate system scaled between zero and one along each axis. In an additional implementation, the coordinates employ a geographic coordinate system such as latitude and longitude values when the facility location permits such precision.

At 340, the polygon generation may employ various geometric construction approaches. In one embodiment, the processor generates simple convex polygons wherein all interior angles are less than 180 degrees. In another embodiment, the processor supports concave polygon generation to accommodate irregularly shaped rooms or areas with alcoves and protrusions. In a further embodiment, the processor enables generation of polygons with interior holes to represent areas containing excluded regions such as elevator shafts or structural columns. In yet another embodiment, the processor supports multi-part polygons comprising disconnected regions that collectively represent a single logical location area.

At 340, the association between polygons and tracking beacons may be established through various mechanisms. In one implementation, the processor automatically associates a polygon with beacons whose stored positions fall within the polygon boundary through point-in-polygon computational analysis. In another implementation, the operator manually selects beacon identifiers from a list to associate with each defined polygon. In a further implementation, the processor associates polygons with beacons based on proximity calculations, linking each polygon to the nearest beacon or beacons within a threshold distance. In an additional implementation, the association utilizes beacon signal strength data collected during a calibration phase to determine which beacons provide coverage for each polygon region.

At 350, the processor assigns a location category or classification to each polygon by associating polygons with corresponding zone, floor, room, hallway, or exterior location identifiers through drag-and-drop user interface input, and stores the associations in the datastore including keep-away zone designations defining areas where specified user devices should not be present.

At 350, the system performs a polygon classification operation wherein the processor executes instructions to categorize spatial boundary definitions according to predetermined location taxonomies. The processor receives polygon data structures representing discrete spatial regions within a monitored environment and facilitates the assignment of semantic location identifiers to each polygon through an interactive graphical user interface mechanism.

At 350, the functionality encompasses receiving user-initiated drag-and-drop interactions wherein location category labels or classification tokens are manipulated within the graphical user interface and deposited onto corresponding polygon representations displayed on a visual rendering of the spatial environment. The processor detects the drag-and-drop event, identifies the source location category being transferred, determines the target polygon receiving the classification, and generates an association record linking the polygon identifier to the selected location category.

At 350, the location categories available for assignment include zone identifiers representing logical groupings of spaces, floor identifiers indicating vertical positioning within multi-story structures, room identifiers specifying enclosed functional spaces, hallway identifiers denoting transitional corridors, and exterior identifiers marking outdoor or peripheral regions. Each category type maintains distinct attributes and behavioral parameters that govern how the system interprets device presence within the classified polygon boundaries.

At 350, the processor further enables designation of keep-away zones through the classification interface, wherein specific polygons receive special restriction flags indicating that particular user devices should not enter or remain within the defined spatial boundaries. The keep-away zone designation includes association data specifying which user devices or device categories are subject to the spatial restriction, enabling granular control over permitted device-location combinations.

At 350, upon completion of each classification assignment, the processor writes the polygon-to-category association to the datastore, creating persistent records that include the polygon identifier, the assigned location category type, the specific location identifier within that category, any keep-away zone designations, lists of restricted device identifiers, and timestamp metadata indicating when the classification was established or modified.

At 350, alternative implementations for the classification assignment mechanism include keyboard-based selection interfaces wherein users navigate through polygon lists and category menus using keystroke commands rather than drag-and-drop gestures. Another alternative involves touch-based tap-and-hold interactions on touch-enabled displays wherein users select polygons through sustained contact and assign categories through secondary menu selections. Voice-command interfaces represent another alternative wherein users verbally identify polygon names and speak corresponding category assignments that are processed through speech recognition modules. Automated classification alternatives include machine learning classifiers that analyze polygon geometric properties, spatial relationships, and contextual features to suggest or automatically assign location categories without direct user manipulation. Batch import mechanisms provide another alternative wherein classification assignments are defined in external configuration files using structured data formats and imported into the system through file upload operations. Template-based classification alternatives enable users to apply predefined classification schemes from previously configured environments to new polygon sets through pattern matching algorithms that identify corresponding spatial structures.

At 360, the processor generates designated beacon location coordinates relative to the global origin, wherein the beacon locations are designated such that ultra-wide band beacons installed at said locations are operable to provide wireless communication coverage with wearable user devices located anywhere within the facility floor plan extent.

The operation at 360 involves computational determination of spatial positions within the facility where ultra-wide band (UWB) beacon hardware should be physically installed to achieve comprehensive wireless signal coverage throughout the entirety of the mapped floor plan area. The processor executes algorithms that analyze the previously established facility geometry and spatial characteristics to output a set of three-dimensional coordinate values, each coordinate value representing a recommended installation point for a UWB beacon device.

At 360, the processor performs coverage analysis calculations that account for the propagation characteristics of ultra-wide band radio signals, including signal attenuation over distance, penetration through structural elements such as walls and partitions identified within the floor plan data, and multipath reflection effects that may occur within the indoor environment. The processor evaluates the floor plan extent boundaries and internal structural features to identify regions requiring beacon coverage and computes beacon placement coordinates that collectively provide overlapping signal coverage zones spanning the complete floor plan area.

The coordinate generation at 360 utilizes the global origin reference point established during prior processing operations, expressing each beacon location as a coordinate triplet (x, y, z) measured from this common reference frame. This coordinate system consistency enables subsequent installation procedures to accurately position physical beacon hardware at the computed locations using measurement references tied to the same global origin.

At 360, the processor may implement various computational approaches for determining beacon placement coordinates. In one embodiment, the processor applies geometric coverage algorithms that model UWB signal range as spherical or ellipsoidal coverage volumes and computes beacon positions using sphere-packing or set-covering optimization techniques to minimize the number of beacons while maintaining complete floor plan coverage. In another embodiment, the processor employs ray-tracing simulation methods that model signal propagation paths through the facility geometry, accounting for material-specific attenuation coefficients associated with walls, floors, and other structural elements to generate beacon coordinates that compensate for signal degradation effects.

At 360, the processor may alternatively utilize grid-based sampling approaches wherein the floor plan extent is discretized into a regular grid of candidate beacon positions, and the processor evaluates coverage metrics for various beacon placement combinations selected from this candidate grid to identify coordinate sets satisfying the coverage requirements. In a further embodiment, the processor may apply machine learning models trained on empirical UWB coverage data from similar facility configurations to predict beacon coordinates likely to achieve adequate coverage performance.

The beacon location coordinates generated at 360 are designated to support positioning functionality for wearable user devices operating within the facility. UWB technology enables precise distance measurements through time-of-flight calculations between beacons and wearable devices, and the beacon placement coordinates are computed such that any location within the floor plan extent maintains line-of-sight or adequate signal connectivity with a sufficient number of beacons to enable triangulation-based position determination for the wearable devices.

At 360, the processor may incorporate constraints reflecting practical installation considerations when generating beacon coordinates, including preferences for mounting locations on walls or ceilings, avoidance of positions that would interfere with facility operations, and alignment with existing electrical infrastructure for power supply connections. The processor may weight candidate positions according to installation feasibility factors while maintaining the coverage completeness requirement across the floor plan extent.

At 370, the processor displays the generated polygons overlaid on the floor plan image with classification or category labels, displays beacon location indicators at designated coordinates, and renders real-time visual indicators for each wearable user device location determined through beacon signal triangulation, enabling facility-wide location monitoring through the graphical user interface.

At 370, the system performs a composite visualization operation that integrates multiple graphical layers to present a unified facility monitoring interface. The processor retrieves the previously generated polygon data structures and renders these geometric shapes as overlay elements positioned on the underlying floor plan image. Each polygon is displayed with its associated classification or category label, which may be rendered as text annotations positioned within or adjacent to the polygon boundaries, or alternatively displayed as color-coded fills, hatching patterns, or other visual differentiation schemes that correspond to the assigned classification categories.

At 370, the processor simultaneously retrieves beacon location data from the system database and generates beacon location indicators at the designated coordinate positions on the floor plan visualization. These beacon indicators may comprise graphical icons, geometric markers such as circles or triangles, or other symbolic representations that identify the physical placement of beacon transmitters throughout the facility. The coordinate positions for beacon placement correspond to the actual installed locations of beacon hardware within the physical environment, with the graphical indicators scaled and positioned according to the coordinate system established for the floor plan image.

At 370, the processor further implements real-time rendering of visual indicators representing the current location of each wearable user device within the monitored facility. The location data for these wearable devices is derived through beacon signal triangulation, wherein the processor analyzes signal strength measurements, time-of-arrival data, or other ranging parameters received from multiple beacon sources to calculate the estimated position of each wearable device. The triangulation computation may employ trilateration algorithms that utilize distance estimates from three or more beacon reference points, or alternatively may implement fingerprinting techniques that compare received signal patterns against a pre-established radio frequency map of the facility.

At 370, the visual indicators for wearable device locations are rendered as dynamic graphical elements that update in response to newly calculated position data. These indicators may comprise avatar representations, geometric markers, identification labels, or other graphical symbols that distinguish individual wearable devices or their associated users. The rendering operation may incorporate animation effects, trailing indicators showing movement history, or directional arrows indicating detected movement vectors.

At 370, the composite visualization is presented through the graphical user interface, which may be implemented as a web-based application rendered within a browser environment, a native desktop application, a mobile application, or a dedicated monitoring terminal interface. The graphical user interface may provide interactive capabilities including pan and zoom functionality for navigating the floor plan, selection mechanisms for querying individual polygons or device indicators, and filtering controls for adjusting the displayed information layers.

At 370, alternative implementations for the polygon overlay rendering may include vector graphics rendering using scalable vector graphics format, rasterized bitmap overlay generation, or three-dimensional perspective rendering that presents the floor plan and polygons in an isometric or oblique projection view. The polygon labels may alternatively be displayed through tooltip popups activated by cursor hover events, through a separate legend panel that correlates colors or patterns with classification categories, or through audio announcements triggered by selection events.

At 370, alternative implementations for beacon location indication may include heat map visualizations showing beacon signal coverage areas, range circle overlays indicating the effective transmission radius of each beacon, or network topology diagrams showing beacon interconnection relationships. The beacon indicators may alternatively be displayed only upon user request through a layer toggle control, or may be rendered with varying opacity or size based on beacon operational status or signal quality metrics.

At 370, alternative implementations for wearable device location rendering may include cluster visualization techniques that aggregate nearby devices into grouped indicators when zoom level prevents individual differentiation, density heat maps showing concentration of devices across facility regions, or tabular list views that present location data in textual format alongside the graphical floor plan. The triangulation methodology may alternatively employ angle-of-arrival calculations using directional antenna arrays, time-difference-of-arrival measurements requiring synchronized beacon timing, or hybrid approaches combining multiple ranging techniques to improve position estimate accuracy.

At 370, the real-time update mechanism for device location indicators may be implemented through polling-based refresh cycles at configurable intervals, through push notification architectures that trigger display updates upon position change events, or through streaming data connections that provide continuous position telemetry. The graphical user interface may alternatively present historical playback functionality enabling review of past device movements, predictive path visualization based on movement pattern analysis, or alert highlighting that emphasizes devices entering or exiting designated polygon regions.

### Hardware Architecture

Generally, the techniques disclosed herein may be implemented on hardware or a combination of software and hardware. For example, they may be implemented in an operating system kernel, in a separate user process, in a library package bound into network applications, on a specially constructed machine, on an application-specific integrated circuit (ASIC), or on a network interface card.

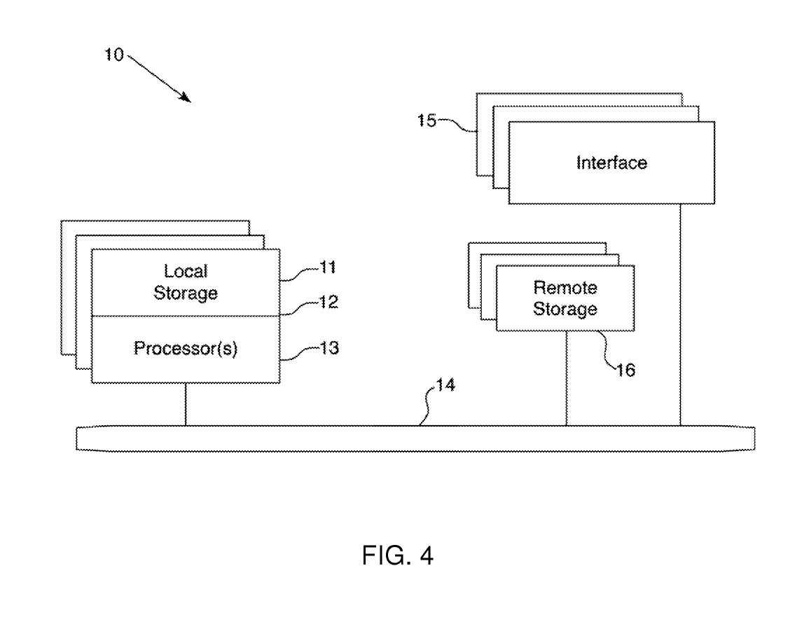

Software/hardware hybrid implementations of at least some of the embodiments disclosed herein may be implemented on a programmable network-resident machine (which should be understood to include intermittently connected network-aware machines) selectively activated or reconfigured by a computer program stored in memory. Such network devices may have multiple network interfaces that may be configured or designed to utilize different types of network communication protocols. A general architecture for some of these machines may be described herein in order to illustrate one or more exemplary means by which a given unit of functionality may be implemented. According to specific embodiments, at least some of the features or functionalities of the various embodiments disclosed herein may be implemented on one or more general-purpose computers associated with one or more networks, such as for example an end-user computer system, a client computer, a network server or other server system, a mobile computing device (e.g., tablet computing device, mobile phone, smartphone, laptop, or other appropriate computing device), a consumer electronic device, a music player, or any other suitable electronic device, router, switch, or other suitable device, or any combination thereof. In at least some embodiments, at least some of the features or functionalities of the various embodiments disclosed herein may be implemented in one or more virtualized computing environments (e.g., network computing clouds, virtual machines hosted on one or more physical computing machines, or other appropriate virtual environments). Any of the above mentioned systems, units, modules, engines, controllers, components, process steps or the like may be and/or comprise hardware and/or software as described herein. For example, the systems, engines, and subcomponents described herein may be and/or comprise computing hardware and/or software as described herein in association with FIGS. 4-7. Furthermore, any of the above mentioned systems, units, modules, engines, controllers, components, interfaces or the like may use and/or comprise an application programming interface (API) for communicating with other systems units, modules, engines, controllers, components, interfaces or the like for obtaining and/or providing data or information.

Referring now to FIG. 4, there is shown a block diagram depicting an exemplary computing device 10 suitable for implementing at least a portion of the features or functionalities disclosed herein. Computing device 10 may be, for example, any one of the computing machines listed in the previous paragraph, or indeed any other electronic device capable of executing software- or hardware-based instructions according to one or more programs stored in memory. Computing device 10 may be configured to communicate with a plurality of other computing devices, such as clients or servers, over communications networks such as a wide area network a metropolitan area network, a local area network, a wireless network, the Internet, or any other network, using known protocols for such communication, whether wireless or wired.

In one aspect, computing device 10 includes one or more central processing units (CPU) 12, one or more interfaces 15, and one or more busses 14 (such as a peripheral component interconnect (PCI) bus). When acting under the control of appropriate software or firmware, CPU 12 may be responsible for implementing specific functions associated with the functions of a specifically configured computing device or machine. For example, in at least one aspect, a computing device 10 may be configured or designed to function as a server system utilizing CPU 12, local memory 11 and/or remote memory 16, and interface(s) 15. In at least one aspect, CPU 12 may be caused to perform one or more of the different types of functions and/or operations under the control of software modules or components, which for example, may include an operating system and any appropriate applications software, drivers, and the like.

CPU 12 may include one or more processors 13 such as, for example, a processor from one of the Intel, ARM, Qualcomm, and AMD families of microprocessors. In some embodiments, processors 13 may include specially designed hardware such as application-specific integrated circuits (ASICs), electrically erasable programmable read-only memories (EEPROMs), field-programmable gate arrays (FPGAs), and so forth, for controlling operations of computing device 10. In a particular aspect, a local memory 11 (such as non-volatile random-access memory (RAM) and/or read-only memory (ROM), including for example one or more levels of cached memory) may also form part of CPU 12. However, there are many different ways in which memory may be coupled to system 10. Memory 11 may be used for a variety of purposes such as, for example, caching and/or storing data, programming instructions, and the like. It should be further appreciated that CPU 12 may be one of a variety of system-on-a-chip (SOC) type hardware that may include additional hardware such as memory or graphics processing chips, such as a QUALCOMM SNAPDRAGON™ or SAMSUNG EXYNOS™ CPU as are becoming increasingly common in the art, such as for use in mobile devices or integrated devices.

As used herein, the term “processor” is not limited merely to those integrated circuits referred to in the art as a processor, a mobile processor, or a microprocessor, but broadly refers to a microcontroller, a microcomputer, a programmable logic controller, an application-specific integrated circuit, and any other programmable circuit.

In one aspect, interfaces 15 are provided as network interface cards (NICs). Generally, NICs control the sending and receiving of data packets over a computer network; other types of interfaces 15 may for example support other peripherals used with computing device 10. Among the interfaces that may be provided are Ethernet interfaces, frame relay interfaces, cable interfaces, DSL interfaces, token ring interfaces, graphics interfaces, and the like. In addition, various types of interfaces may be provided such as, for example, universal serial bus (USB), Serial, Ethernet, FIREWIRE™, THUNDERBOLT™, PCI, parallel, radio frequency (RF), BLUETOOTH™, near-field communications (e.g., using near-field magnetics), 802.11 (WiFi), frame relay, TCP/IP, ISDN, fast Ethernet interfaces, Gigabit Ethernet interfaces, Serial ATA (SATA) or external SATA (ESATA) interfaces, high-definition multimedia interface (HDMI), digital visual interface (DVI), analog or digital audio interfaces, asynchronous transfer mode (ATM) interfaces, high-speed serial interface (HSSI) interfaces, Point of Sale (POS) interfaces, fiber data distributed interfaces (FDDIs), and the like. Generally, such interfaces 15 may include physical ports appropriate for communication with appropriate media. In some cases, they may also include an independent processor (such as a dedicated audio or video processor, as is common in the art for high-fidelity A/V hardware interfaces) and, in some instances, volatile and/or non-volatile memory (e.g., RAM).

Although the system shown in FIG. 4 illustrates one specific architecture for a computing device 10 for implementing one or more of the embodiments described herein, it is by no means the only device architecture on which at least a portion of the features and techniques described herein may be implemented. For example, architectures having one or any number of processors 13 may be used, and such processors 13 may be present in a single device or distributed among any number of devices. In one aspect, single processor 13 handles communications as well as routing computations, while in other embodiments a separate dedicated communications processor may be provided. In various embodiments, different types of features or functionalities may be implemented in a system according to the aspect that includes a client device (such as a tablet device or smartphone running client software) and server systems (such as a server system described in more detail below).

Regardless of network device configuration, the system of an aspect may employ one or more memories or memory modules (such as, for example, remote memory block 16 and local memory 11) configured to store data, program instructions for the general-purpose network operations, or other information relating to the functionality of the embodiments described herein (or any combinations of the above). Program instructions may control execution of or comprise an operating system and/or one or more applications, for example. Memory 16 or memories 11, 16 may also be configured to store data structures, configuration data, encryption data, historical system operations information, or any other specific or generic non-program information described herein.

Because such information and program instructions may be employed to implement one or more systems or methods described herein, at least some network device embodiments may include nontransitory machine-readable storage media, which, for example, may be configured or designed to store program instructions, state information, and the like for performing various operations described herein. Examples of such nontransitory machine- readable storage media include, but are not limited to, magnetic media such as hard disks, floppy disks, and magnetic tape; optical media such as CD-ROM disks; magneto-optical media such as optical disks, and hardware devices that are specially configured to store and perform program instructions, such as read-only memory devices (ROM), flash memory (as is common in mobile devices and integrated systems), solid state drives (SSD) and “hybrid SSD” storage drives that may combine physical components of solid state and hard disk drives in a single hardware device (as are becoming increasingly common in the art with regard to personal computers), memristor memory, random access memory (RAM), and the like. It should be appreciated that such storage means may be integral and non-removable (such as RAM hardware modules that may be soldered onto a motherboard or otherwise integrated into an electronic device), or they may be removable such as swappable flash memory modules (such as “thumb drives” or other removable media designed for rapidly exchanging physical storage devices), “hot-swappable” hard disk drives or solid state drives, removable optical storage discs, or other such removable media, and that such integral and removable storage media may be utilized interchangeably. Examples of program instructions include both object code, such as may be produced by a compiler, machine code, such as may be produced by an assembler or a linker, byte code, such as may be generated by for example a JAVA™ compiler and may be executed using a Java virtual machine or equivalent, or files containing higher level code that may be executed by the computer using an interpreter (for example, scripts written in Python, Perl, Ruby, Groovy, or any other scripting language).

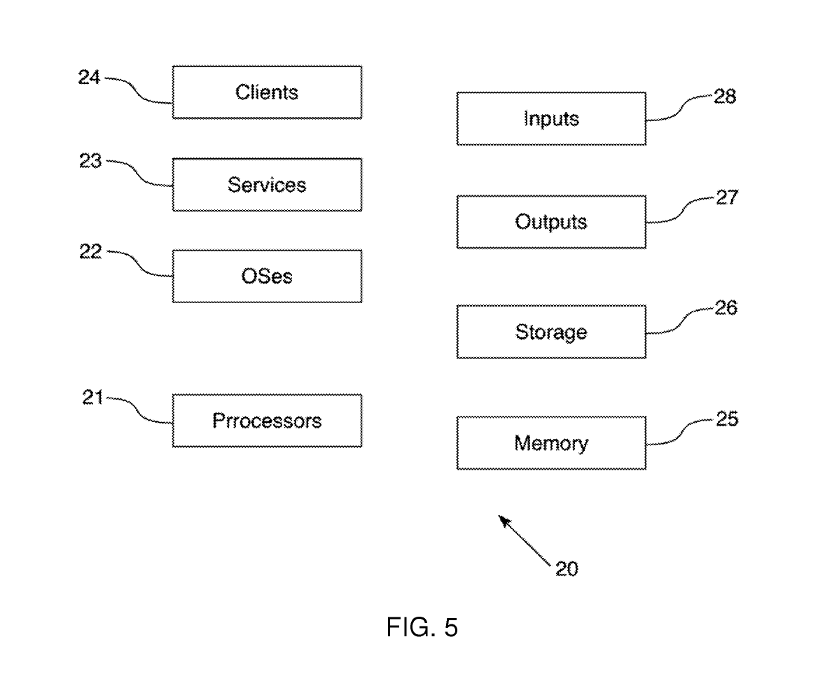

In some embodiments, systems may be implemented on a standalone computing system. Referring now to FIG. 5, there is shown a block diagram depicting a typical exemplary architecture of one or more embodiments or components thereof on a standalone computing system. Computing device 20 includes processors 21 that may run software that carry out one or more functions or applications of embodiments, such as for example a client application. Processors 21 may carry out computing instructions under control of an operating system 22 such as, for example, a version of MICROSOFT WINDOWS™ operating system, APPLE macOS™ or iOS™ operating systems, some variety of the Linux operating system, ANDROID™ operating system, or the like. In many cases, one or more shared services 23 may be operable in system 20, and may be useful for providing common services to client applications. Services 23 may for example be WINDOWS™ services, user-space common services in a Linux environment, or any other type of common service architecture used with operating system 21. Input devices 28 may be of any type suitable for receiving user input, including for example a keyboard, touchscreen, microphone (for example, for voice input), mouse, touchpad, trackball, or any combination thereof. Output devices 27 may be of any type suitable for providing output to one or more users, whether remote or local to system 20, and may include for example one or more screens for visual output, speakers, printers, or any combination thereof. Memory 25 may be random-access memory having any structure and architecture known in the art, for use by processors 21, for example to run software. Storage devices 26 may be any magnetic, optical, mechanical, memristor, or electrical storage device for storage of data in digital form (such as those described above, referring to FIG. 4). Examples of storage devices 26 include flash memory, magnetic hard drive, CD-ROM, and/or the like.

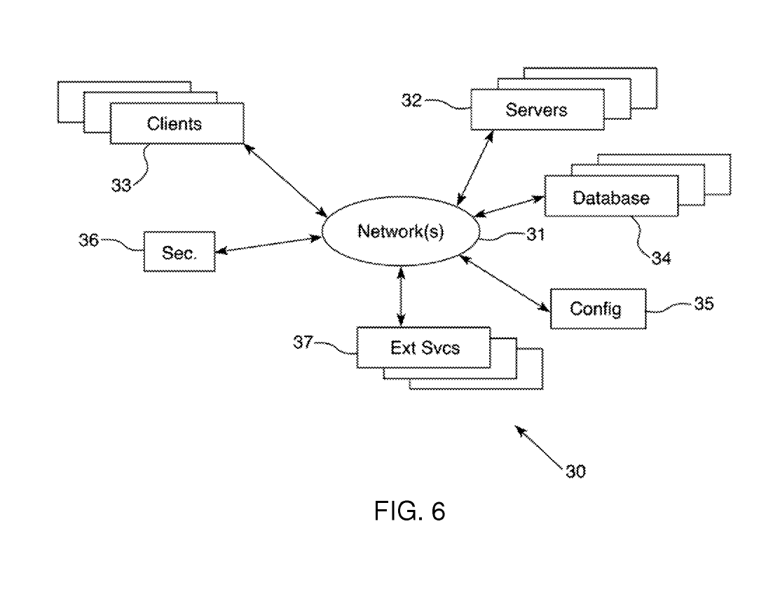

In some embodiments, systems may be implemented on a distributed computing network, such as one having any number of clients and/or servers. Referring now to FIG. 6, there is shown a block diagram depicting an exemplary architecture 30 for implementing at least a portion of a system according to one aspect on a distributed computing network. According to the aspect, any number of clients 33 may be provided. Each client 33 may run software for implementing client-side portions of a system; clients may comprise a system 20 such as that illustrated in FIG. 5. In addition, any number of servers 32 may be provided for handling requests received from one or more clients 33. Clients 33 and servers 32 may communicate with one another via one or more electronic networks 31, which may be in various embodiments any of the Internet, a wide area network, a mobile telephony network (such as CDMA or GSM cellular networks), a wireless network (such as WiFi, WiMAX, LTE, and so forth), or a local area network (or indeed any network topology known in the art; the aspect does not prefer any one network topology over any other). Networks 31 may be implemented using any known network protocols, including for example wired and/or wireless protocols.

In addition, in some embodiments, servers 32 may call external services 37 when needed to obtain additional information, or to refer to additional data concerning a particular call. Communications with external services 37 may take place, for example, via one or more networks 31. In various embodiments, external services 37 may comprise web-enabled services or functionality related to or installed on the hardware device itself. For example, in one aspect where client applications are implemented on a smartphone or other electronic device, client applications may obtain information stored in a server system 32 in the cloud or on an external service 37 deployed on one or more of a particular enterprise’s or user’s premises.

In some embodiments, clients 33 or servers 32 (or both) may make use of one or more specialized services or appliances that may be deployed locally or remotely across one or more networks 31. For example, one or more databases 34 may be used or referred to by one or more embodiments. It should be understood by one having ordinary skill in the art that databases 34 may be arranged in a wide variety of architectures and using a wide variety of data access and manipulation means. For example, in various embodiments one or more databases 34 may comprise a relational database system using a structured query language (SQL), while others may comprise an alternative data storage technology such as those referred to in the art as “NoSQL” (for example, HADOOP CASSANDRA™, GOOGLE BIGTABLE™, and so forth). In some embodiments, variant database architectures such as column-oriented databases, in-memory databases, clustered databases, distributed databases, or even flat file data repositories may be used according to the aspect. It will be appreciated by one having ordinary skill in the art that any combination of known or future database technologies may be used as appropriate, unless a specific database technology or a specific arrangement of components is specified for a particular aspect described herein. Moreover, it should be appreciated that the term “database” as used herein may refer to a physical database machine, a cluster of machines acting as a single database system, or a logical database within an overall database management system. Unless a specific meaning is specified for a given use of the term “database”, it should be construed to mean any of these senses of the word, all of which are understood as a plain meaning of the term “database” by those having ordinary skill in the art.

Similarly, some embodiments may make use of one or more security systems 36 and configuration systems 35. Security and configuration management are common information technology (IT) and web functions, and some amount of each are generally associated with any IT or web systems. It should be understood by one having ordinary skill in the art that any configuration or security subsystems known in the art now or in the future may be used in conjunction with embodiments without limitation, unless a specific security 36 or configuration system 35 or approach is specifically required by the description of any specific aspect.

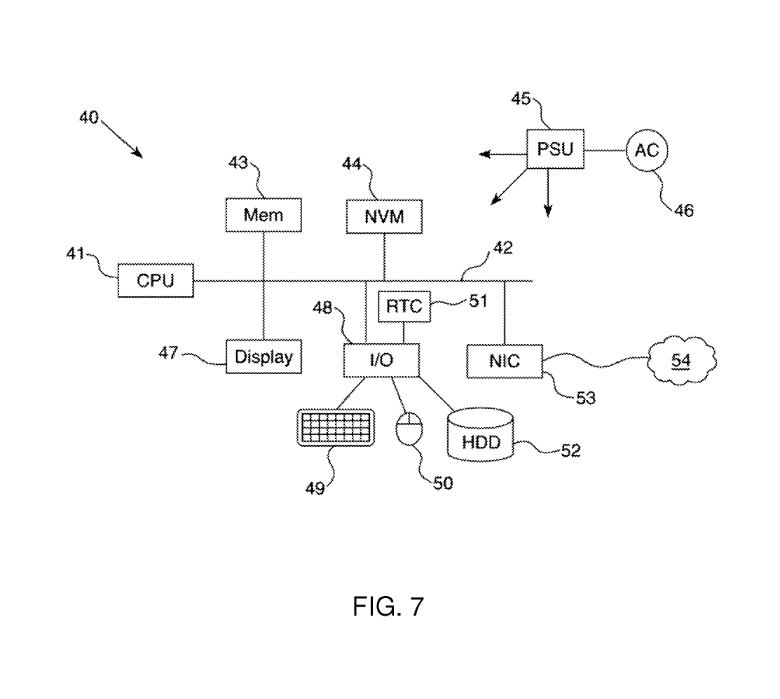

FIG. 7 shows an exemplary overview of a computer system 40 as may be used in any of the various locations throughout the system. It is exemplary of any computer that may execute code to process data. Various modifications and changes may be made to computer system 40 without departing from the broader scope of the system and method disclosed herein. Central processor unit (CPU) 41 is connected to bus 42, to which bus is also connected memory 43, nonvolatile memory 44, display 47, input/output (I/O) unit 48, and network interface card (NIC) 53. I/O unit 48 may, typically, be connected to keyboard 49, pointing device 50, hard disk 52, and real-time clock 51. NIC 53 connects to network 54, which may be the Internet or a local network, which local network may or may not have connections to the Internet. Also shown as part of system 40 is power supply unit 45 connected, in this example, to a main alternating current (AC) supply 46. Not shown are batteries that could be present, and many other devices and modifications that are well known but are not applicable to the specific novel functions of the current system and method disclosed herein. It should be appreciated that some or all components illustrated may be combined, such as in various integrated applications, for example Qualcomm or Samsung system-on-a-chip (SOC) devices, or whenever it may be appropriate to combine multiple capabilities or functions into a single hardware device (for instance, in mobile devices such as smartphones, video game consoles, in-vehicle computer systems such as navigation or multimedia systems in automobiles, or other integrated hardware devices).

In various embodiments, functionality for implementing systems or methods of various embodiments may be distributed among any number of client and/or server components. For example, various software modules may be implemented for performing various functions in connection with the system of any particular aspect, and such modules may be variously implemented to run on server and/or client components.

The skilled person will be aware of a range of possible modifications of the various embodiments described above. Accordingly, the present invention is defined by the claims and their equivalents.

### Additional Considerations

As used herein any reference to "one embodiment" or "an embodiment" means that a particular element, feature, structure, or characteristic described in connection with the embodiment is included in at least one embodiment. The appearances of the phrase "in one embodiment" in various places in the specification are not necessarily all referring to the same embodiment.

Some embodiments may be described using the expression "coupled" and "connected" along with their derivatives. For example, some embodiments may be described using the term "coupled" to indicate that two or more elements are in direct physical or electrical contact. The term "coupled," however, may also mean that two or more elements are not in direct contact with each other, but yet still co-operate or interact with each other. The embodiments are not limited in this context.

As used herein, the terms "comprises," "comprising," "includes," "including," "has," "having" or any other variation thereof, are intended to cover a non-exclusive inclusion. For example, a process, method, article, or apparatus that comprises a list of elements is not necessarily limited to only those elements but may include other elements not expressly listed or inherent to such process, method, article, or apparatus. Further, unless expressly stated to the contrary, "or" refers to an inclusive or and not to an exclusive or. For example, a condition A or B is satisfied by any one of the following: A is true (or present) and Bis false (or not present), A is false ( or not present) and Bis true ( or present), and both A and B are true ( or present).

In addition, use of the "a" or "an" are employed to describe elements and components of the embodiments herein. This is done merely for convenience and to give a general sense of the invention. This description should be read to include one or at least one and the singular also includes the plural unless it is obvious that it is meant otherwise.

Upon reading this disclosure, those of skill in the art will appreciate still additional alternative structural and functional designs for a system and/or a process associated with the disclosed principles herein. Thus, while particular embodiments and applications have been illustrated and described, it is to be understood that the disclosed embodiments are not limited to the precise construction and components disclosed herein. Various apparent modifications, changes and variations may be made in the arrangement, operation and details of the method and apparatus disclosed herein without departing from the spirit and scope defined in the appended claims.

What is claimed is:

1. A computer implemented method for generating a mapped floor plan for determining user device locations relative to the floor plan, the computer implemented method comprising:
obtaining floor plan data by importing a file associated with a floor plan of a facility, wherein the floor plan indicates at least one of a zone, floor, room, hallway and exterior location associated with the facility;
defining a global origin by assigning a first set of coordinates within the floor plan based on the imported file;
assigning a second set of coordinates within the floor plan to generate at least one polygon associated with the second set of coordinates wherein the at least one polygon defines at least one location area, wherein each location area is associated with at least one tracking beacon, wherein each polygon is associated with a zone, floor, room, hallway or exterior location associated with the facility;
assigning a location category or classification to each polygon, wherein assigning comprises associating the polygons with a corresponding zone, floor, room, hallway or exterior location indicated by the floor plan and storing the association in a datastore;
generating designated beacon locations, wherein the beacon locations are designated such that the beacons are operable to provide wireless communication with wearable user devices located within the facility;
displaying the polygons overlaid on the floor plan and with the classification or category label; and
displaying the beacon locations overlaid on the floor plan.

2. The computer implemented method according to claim 1, wherein the wearable user devices comprise a smart wearable or smart watch.

3. The computer implemented method according to claim 1, wherein the imported file comprises an image file associated with a floor plan for a building.

4. The computer implemented method according to claim 1, further comprising installing beacons at the facility in accordance with the generated beacon locations.

5. The computer implemented method according to claim 1, wherein the beacons comprise ultra wide band beacons.

6. The computer implemented method according to claim 1, wherein the polygons are generated via input from a user interface (UI), wherein the UI allows a user to provide polygon information.

7. The computer implemented method according to claim 1, wherein the beacons are configured to communicate with user devices at periodic time intervals to generate a set of historical locations of the user device.

8. The computer implemented method according to claim 7, wherein the set of historical locations comprises data associated with at least one of the coordinates of the user device, at least one beacon used in determining the device location, and a zone, floor, room, hallway or exterior location associated with the floor plan.

9. The computer implemented method according to claim 1, wherein the beacons are configured to communicate with user devices at periodic time intervals to generate periodic head counts of users housed in the facility.

10. The computer implemented method according to claim 1, wherein the beacons are configured to communicate with user devices upon receiving a command such that a real-time location of a user device can be determined in response to the command.

11. The computer implemented method according to claim 1, further comprising displaying a visual indicator for each user device location relative to the floor plan.

12. The computer implemented method according to claim 1, wherein assigning a location category or classification comprises obtaining, via a user interface, input indicative of the category or classification.

13. The computer implemented method according to claim 1, wherein the category or classification comprises identifying floor plan areas where user devices should not be present, are allowed to be present, or have limited or special access requirements.

14. The computer implemented method according to claim 1, wherein assigning a location category or classification comprises assigning keep away zones, wherein the keep away zones define areas where at least one user device should not be present.

15. The computer implemented method according to claim 14, wherein the keep away zones comprise keep away restrictions associated with the proximity of two or more user devices.

16. The computer implemented method according to claim 1, further comprising calibrating the beacons after beacons have been placed at the designated beacon locations in order to allow for accurate triangulation or other position determination.

17. The computer implemented method according to claim 1, further comprising obtaining by at least one beacon, a signal from a user device; processing the signal obtained by the at least one beacon to determine a corresponding user device location; and displaying on a graphical user interface a location indicator associated with the user device, thereby indicating the location of the user device relative to the floor plan of the facility.

18. The computer implemented method according to claim 1, wherein beacon locations comprise coordinates relative to the global origin indicating the location of each beacon.

19. The computer implemented method according to claim 1, wherein beacon locations comprise an arrangement such that the beacons cover the extent of the floor plan such that any location of a user device within an area corresponding to the floor plan can be determined.

20. The computer implemented method according to claim 1, further comprising obtaining location data for a plurality of different user devices, thereby providing real-time headcount and location data for individuals associated with a facility.

21. A computing system for generating a mapped floor plan for determining user device locations relative to the floor plan, the computing system comprising:
at least one computing processor; and
memory comprising instructions that, when executed by the at least one computing processor, enable the computing system to execute a process comprising:
obtaining floor plan data by importing a file associated with a floor plan of a facility, wherein the floor plan indicates at least one of a zone, floor, room, hallway and exterior location associated with the facility;
defining a global origin by assigning a first set of coordinates within the floor plan based on the imported file;
assigning a second set of coordinates within the floor plan to generate at least one polygon associated with the second set of coordinates wherein the at least one polygon defines at least one location area, wherein each location area is associated with at least one tracking beacon, wherein each polygon is associated with a zone, floor, room, hallway or exterior location associated with the facility;
assigning a location category or classification to each polygon, wherein assigning comprises associating the polygons with a corresponding zone, floor, room, hallway or exterior location indicated by the floor plan and storing the association in a datastore;
generating designated beacon locations, wherein the beacon locations are designated such that the beacons are operable to provide wireless communication with wearable user devices located within the facility;
displaying the polygons overlaid on the floor plan and with the classification or category label; and
displaying the beacon locations overlaid on the floor plan.

22. A non-transitory computer readable medium comprising instructions that when executed by a processor enable the processor to execute a process comprising:
obtaining floor plan data by importing a file associated with a floor plan of a facility, wherein the floor plan indicates at least one of a zone, floor, room, hallway and exterior location associated with the facility;
defining a global origin by assigning a first set of coordinates within the floor plan based on the imported file;
assigning a second set of coordinates within the floor plan to generate at least one polygon associated with the second set of coordinates wherein the at least one polygon defines at least one location area, wherein each location area is associated with at least one tracking beacon, wherein each polygon is associated with a zone, floor, room, hallway or exterior location associated with the facility;
assigning a location category or classification to each polygon, wherein assigning comprises associating the polygons with a corresponding zone, floor, room, hallway or exterior location indicated by the floor plan and storing the association in a datastore;
generating designated beacon locations, wherein the beacon locations are designated such that the beacons are operable to provide wireless communication with wearable user devices located within the facility;
displaying the polygons overlaid on the floor plan and with the classification or category label; and
displaying the beacon locations overlaid on the floor plan.
